(19) United States
(12) Patent Application Publication
(10) Pub. No.: US 2017/0281480 A1
(43) Pub. Date: Oct. 5, 2017

(54) PROCESSES FOR PREPARING MULTIPLE CAPSULES

(71) Applicant: INTERNATIONAL FLAVORS & FRAGRANCES INC., NE YORK, NY (US)

(72) Inventors: Franklin Pringgosusanto, Laurence Harbor, NJ (US); Lewis Michael Popplewell, Morganville, NJ (US); John Brahms, Morris Plains, NJ (US); Yabin Lei, Holmdel, NJ (US); Judith Kerschner, Hawthorne, NJ (US); Michael V. Imperiale, Newark, NJ (US)

(21) Appl. No.: 15/504,766

(22) PCT Filed: Aug. 19, 2015
(86) PCT No.: PCT/US15/45863

§ 371 Date: Feb. 17, 2017

Related U.S. Application Data
(60) Provisional application No. 62/039,252, filed on Aug. 19, 2014.

(51) Int. Cl. A61K 8/11; A61Q 15/00; B01J 13/14; C11D 3/50; C11D 3/00; A61Q 13/00; A61K 8/87

(52) U.S. Cl. CPC A61K 8/11; A61Q 13/00; A61Q 15/00; A61K 8/87; C11D 3/505; C11D 3/001; B01J 13/14; A61K 2800/805

(57) ABSTRACT

Methods of preparing a capsule delivery system having two or more different capsules. Also disclosed are capsule delivery systems and consumer products containing such a capsule delivery system.

### CROSS-REFERENCE TO RELATED APPLICATIONS

This application claims priority to U.S. Patent Application Ser. No. 62/039,252 filed on Aug. 19, 2014, the content of which is incorporated herein by reference in its entirety.

### BACKGROUND

Fragrance materials are used in numerous products to enhance the consumer's enjoyment of a product. Fragrance materials are added to consumer products such as laundry detergents, fabric softeners, soaps, detergents, personal care products, such as shampoos, body washes, deodorants and the like, as well as numerous other products.

To enhance the effectiveness of the fragrance materials for the user, various technologies have been employed to enhance the delivery of the fragrance materials at the desired time. One widely used technology is encapsulation of the fragrance material in a protective coating. Frequently the protective coating is a polymeric material. The polymeric material is used to protect the fragrance material from evaporation, reaction, oxidation or otherwise dissipating prior to use. For example, U.S. Pat. No. 4,081,384 discloses a softener or anti-stat core coated by a polycondensate suitable for use in a fabric conditioner. U.S. Pat. No. 5,112,688 discloses selected fragrance materials having the proper volatility to be coated by coacervation with microparticles in a wall that can be activated for use in fabric conditioning. U.S. Pat. No. 5,145,842 discloses a solid core of a fatty alcohol, ester, or other solid plus a fragrance coated by an aminoplast shell. U.S. Pat. No. 6,248,703 discloses various agents including fragrance in an aminoplast shell that is included in an extruded bar soap. However, current capsule preparation methods do not yield capsule delivery systems having multiple different capsules that provide optimized release profiles.

There is a need for a process to prepare capsule delivery systems having multiple capsules with optimized organoleptic/release profiles.

### SUMMARY

The present invention is based on the discovery of a convenient process for preparing multiple-capsule delivery system. The capsules can be readily optimized to meet different needs in a wide variety of consumer applications.

One aspect of this invention relates to a method of preparing a capsule delivery system having two or more different capsules. The method includes the steps of: (a) providing a first emulsion containing a first benefit agent and a first capsule wall-forming material; (b) providing a second emulsion containing a second benefit agent and a second capsule wall-forming material; (c) mixing the first emulsion and the second emulsion to obtain an emulsion mixture; (d) optionally adding an activation agent to the emulsion mixture; (e) causing the formation of a first capsule and a second capsule to obtain a mixed capsule slurry, in which the first capsule is different from the second capsule, the first capsule contains the first benefit agent encapsulated by a first capsule wall formed of the first capsule wall-forming material, and the second capsule contains the second benefit agent encapsulated by a second capsule wall formed of the second capsule wall-forming material; and (f) curing the mixed capsule slurry (e.g., at 20° C. to 150° C., 20-45° C., 55° C. to 95° C., 95° C. to 130° C., and 75° C. to 110° C.), to obtain the capsule delivery system. Optionally, a catalyst is added to the first emulsion, the second emulsion, or the emulsion mixture. Further, a malodor counteracting agent can be added to capsule delivery system. The method can further include the step of adding a third, fourth, fifth, or sixth emulsion to the emulsion mixture or mixed capsule slurry, and causing the formation of a third, fourth, fifth, or sixth capsule, in which each of these capsules contains a benefit agent encapsulated by a capsule wall.

In some embodiments, the first or second wall, independently, is formed of a polyacrylate, polyurea, polyurethane, polyacrylamide, poly(acrylate-co-acrylamide), starch, silica, gelatin and gum Arabic, poly(melamine-formaldehyde), poly(urea-formaldehyde), or a combination thereof.

In those embodiments, when the first or second wall is formed of a polyurea or polyurethane, the activation agent is an amine cross-linker or an alcohol cross-linker, respectively. The polyurea capsule wall is typically formed by a reaction between a polyisocyanate and an amine cross-linker. The polyisocyanate is an aromatic or aliphatic isocyanate containing two or more isocyanate groups (i.e., —NCO). The amine cross-linker contains two or more amine groups, which are —NH₂ or —NHR— (R being H, alkyl, cycloalkyl, heterocycloalkyl, heteroalkyl, aryl, heteroaryl, and etc.). The polyurethane capsule wall can be formed by a reaction between a polyisocyanate and an alcohol cross-linker, which contains two or more hydroxyl groups (i.e., —OH). In certain embodiments, a hybrid cross-linker is used. The hybrid cross-linker contains one or more amine groups and one or more hydroxyl groups. It reacts with a polyisocyanate to yield a polymer that has both urea (—NRCONH—) and urethane (—NHCOO—) functional groups.

“Alkyl” means a linear saturated monovalent hydrocarbon radical of one to twenty (e.g., 1-10 and 1-6) carbon atoms or a branched saturated monovalent hydrocarbon radical of three to twenty (e.g., 3-10 and 3-6) carbon atoms, e.g., methyl, ethyl, propyl, 2-propyl, butyl (including all isomeric forms), pentyl (including all isomeric forms), hexyl (including all isomeric forms), and the like. “Cycloalkyl” means a monocyclic, fused bicyclic, or fused tricyclic, saturated or unsaturated, monovalent hydrocarbon radical of three to fourteen carbon ring atoms. Unless otherwise stated, the valency of the group may be located on any atom of any ring within the radical, valency rules permitting. More specifically, the term cycloalkyl includes, but is not limited to, cyclopropyl, cyclobutyl, cyclopentyl, cyclohexyl, cycloheptyl, decahydronaphthyl (e.g., decahydronaphth-1-yl, decahydronaphth-2-yl, and the like), norbornyl, adamantly, cyclopropenyl, cyclobutenyl, cyclopentenyl, cyclohexenyl, and the like. The cycloalkyl ring is unsubstituted or may be substituted with one or more “ring system substituents” which may be the same or different, and are as defined herein. “Heterocycloalkyl” means a saturated or unsaturated, nonaromatic, 5-8 membered monocyclic, 8-12 membered bicyclic, or 11-14 membered tricyclic ring system having one or more heteroatoms (e.g., O, N, P, and S). Examples include, but are not limited to, 2-piperidinonyl, 3-piperidonyl, 4-piperidonyl, piperidinyl, piperazinyl, imidazolidinyl, imidazolidonyl, azepanyl, pyrrolidinyl, 2-pyrrolidonyl, 3-pyrrolidonyl, dihydrothiadiazolyl, dioxanyl, morpholinyl, 2-morpholinonyl, 3-morpholinonyl, tetrahydropuranyl, and tetrahydrofuranyl. The term “heterocycloalkylene” refers to bivalent heterocycloalkyl. The term “heterocycloalkenyl” refers to a nonaromatic 5-8 membered monocyclic, 8-12 membered bicyclic, or 11-14 membered tricyclic ring system having one or more heteroatoms (e.g., O, N, P, and S) and one or more double bond. “Heteroalkyl” means an alkyl radical as defined herein where one or more carbon atoms are replaced by an oxygen, nitrogen, phosphorous, or sulphur atom. Examples include an alkoxy group (e.g., methoxy, ethoxy, propoxy, iso-propoxy, butoxy, and tert-butoxy), an alkoxyalkyl group (e.g., methoxymethyl, ethoxymethyl, 1-methoxy-ethyl, 1-ethoxyethyl, 2-methoxyethyl, and 2-ethoxyethyl), an alkylamino group (e.g., methylamino, ethylamino, propylamino, isopropylamino, dimethylamino, and diethylamino), an alkylthio group (e.g., methylthio, ethylthio, and isopropylthio), and a cyano group. Any heteroalkyl group as defined herein may be substituted with one, two or more substituents, for example, F, Cl, Br, I, NH₂, OH, SH, NO₂, cyclohexyl, 2-piperidonyl, 3-piperidonyl, and 4-piperidonyl. “Aryl” means a monovalent, monocyclic, fused bicyclic, or tricyclic hydrocarbon radical of 6 to 14 ring atoms, wherein the ring comprising a monocyclic radical ring is aromatic and wherein at least one of the fused rings containing a bicyclic or tricyclic radical is aromatic. Unless otherwise stated, the valency of the group may be located on any atom of any ring within the radical, valency rules permitting. More specifically, the term aryl includes, but is not limited to, phenyl, naphthyl, anthracenyl, indanyl (including, for example, indan-5-yl, or indan-2-yl, and the like), tetrahydronaphthyl (including, for example, tetrahydronaphth-1-yl, tetrahydronaphth-2-yl, and the like), and the like. Unless indicated otherwise, aryl is unsubstituted or may be substituted with one or more “ring system substituents” which may be the same or different, and are as defined herein. “Heteroaryl” means a monocyclic or fused bicyclic, monovalent radical of 5 to 12 ring atoms containing one or more, preferably one, two, three, or four ring heteroatoms independently selected from the group of N, O, P(O)ₘ, Si (where Si is substituted with alkyl and one additional group selected from alkyl, alkenyl, cycloalkylalkyl, aryl, aralkyl, heteroaralkyl, and optionally substituted heterocycloalkylalkyl), and S(O)ₙ, where m is 1 or 2 and n is 0, 1, or 2, the remaining ring atoms being carbon, wherein the ring comprising a monocyclic radical is aromatic and wherein at least one of the fused rings comprising the bicyclic radical is aromatic. One or two ring carbon atoms can optionally be replaced by a —C(O)—, —C(S)—, or C(═NH)— group. Unless otherwise stated, the valency may be located on any atom of any ring of the heteroaryl group, valency rules permitting. More specifically, the term heteroaryl includes, but is not limited to, phthalimidyl, pyridinyl, pyrrolyl, pyrazolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), pyrazinyl, pyrimidinyl, pyridazinyl, oxazolyl, isoxazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, methylenedioxyphenyl (including, for example, methylenedioxyphen-5-yl), and the derivatives thereof, or N-oxide or a protected derivative thereof. The heteroaryl ring is unsubstituted or may be substituted with one, two, or three “ring system substituents” which may be the same or different, and are as defined herein.

Benefit agents encapsulated in these capsules include, but are not limited to, a fragrance, pro-fragrance, flavor, malodor counteractive agent, anti-inflammatory agent, anesthetic, analgesic, anti-viral agent, anti-bacterial agent, anti-infectious agent, anti-acne agent, skin lightening agent, insect repellant, emollient, skin moisturizing agent, vitamin or derivative thereof, nanometer to micron size inorganic solid, polymeric or elastomeric particle, or combination thereof.

Another aspect of this invention related to a method of preparing a capsule delivery system including the steps of: (a) providing a first capsule slurry including a first capsule, in which the first capsule containing a first benefit agent encapsulated by a first capsule wall that is formed of a first wall-forming material; (b) providing a second emulsion including a second benefit agent and a second wall-forming material; (c) mixing the first capsule slurry and the second emulsion to obtain a capsule mixture; (d) optionally adding an activation agent to the capsule mixture; (e) causing the formation of a second capsule to obtain a mixed capsule slurry, in which the second capsule contains the second benefit agent and a second wall, the second benefit agent is encapsulated by the second wall, and the second wall is formed of the second wall-forming material; and (f) curing the mixed capsule slurry (e.g., at 20-150° C., 55° C. to 130° C., 55° C. to 95° C., 95° C. to 130° C., and 75° C. to 110° C.), to obtain the capsule delivery system. The first and second capsules are different. The first and second benefit agents, the first and second wall-forming materials, and the activation agent are described above. A catalyst can be added to assist wall-forming reactions between the wall-forming material and the activation agent. Further, before step (c), the first capsule is optionally cured to obtain a cured first capsule. A quenching agent can be added to the cured first capsule to stop further wall-forming reactions. A malodor counteracting agent can also be added to the capsule delivery system.

The method can also include the step of adding a third, fourth, fifth, or sixth emulsion to the capsule mixture or mixed capsule slurry, and causing the formation of a third, fourth, fifth, or sixth capsule, in which each of these capsules contains a benefit agent encapsulated by a capsule wall.

Also within the scope of this invention is a method of preparing a capsule delivery system containing two or more different capsules including the steps of: (a) providing a first capsule slurry including a water phase and a first capsule, in which the first capsule contains a first core material and a first capsule wall formed of a first wall-forming material, and the first core material has a first benefit agent; (b) adding a second core material and a second wall-forming material to the first capsule slurry, in which the second core material has a second benefit agent; (c) emulsifying the second core material into the water phase; (d) causing the formation of a second capsule to obtain a mixed capsule slurry, in which the second capsule contains the second benefit agent encapsulated by a second wall, the second wall is formed of the second wall-forming material, and the second capsule is different from the first capsule; and (e) curing the mixed capsule slurry, e.g., at 55° C. to 130° C. (55° C. to 95° C., 95° C. to 130° C., and 75° C. to 110° C.), to obtain the capsule delivery system. The first and second capsules are different. The first or second capsule wall and wall-forming materials, and the first and second benefit agents are described above.

In some embodiments, the method further includes, before step (c), curing the first capsule slurry to obtain a cured first capsule. A quenching agent can be added to the first capsule slurry to stop further polymerization. Furthermore, a catalyst can also be added into the first capsule slurry, or the second emulsion. Optionally, a malodor counteracting agent is added to the capsule delivery system. In other embodiments, the method further include the step of adding a third, fourth, fifth, or sixth emulsion to the first capsule emulsion, the second capsule emulsion, or the mixed capsule slurry, and causing the formation of a third, fourth, fifth, or sixth capsule, in which each of these capsules contains a benefit agent encapsulated by a capsule wall.

Each of the capsules descried above can have a size of 0.01 to 1000 microns (e.g., 0.1 to 500 microns, 1 to 100 microns, 5 to 200 microns).

Still within the scope of this invention are capsule delivery systems prepared by any of the methods described above. Yet within the scope of the invention are a personal care product, beauty care product, fabric care product, home care product, and oral care product, each of which contains one of these capsule delivery systems.

The details of one or more embodiments of the invention are set forth in the description below. Other features, objects, and advantages of the invention will be apparent from the description and the claims.

### DETAILED DESCRIPTION OF THE INVENTION

A “one-pot” synthetic process is described herein for preparing a capsule delivery system including two or more (e.g., three, four, five, and six) different capsules, each of which contains a benefit agent.

In one method of this invention, each of the capsules is formed in one reactor. In accordance with this embodiment, a first and second emulsions are provided. The first and second emulsions can be prepared in separate reactors.

To prepare a first emulsion, a first oil phase and a first water phase are mixed in a first reactor. Typically, the first oil phase contains a first benefit agent and a first wall-forming material, and the first water phase contains a first dispersant and water. The first oil phase is then emulsified into the first water phase under high shearing at a rate of 3,000 to 20,000 rpm (e.g., 6,000 to 15,000 rpm) to form a first capsule emulsion having a plurality of first oil droplets dispersed in the first capsule emulsion (i.e., an oil-in-water emulsion). The first oil droplets thus formed each have a size of 0.01 to 1000 microns depending on the shear rate, dispersant and its concentration, the benefit agent and its concentration, the ratio of the oil phase to the water phase, and etc. Alternatively, the first benefit agent is included in the water phase and the resultant first capsule emulsion is a water-in-oil emulsion.

A second emulsion can be prepared following the same procedure as the first emulsion in a separate reactor. More specifically, a second oil phase containing a second benefit agent and a second wall-forming material is mixed with a second water phase containing a second dispersant and water. The second oil phase is then emulsified into the second water phase under high shearing at a rate of 3,000 to 20,000 rpm (e.g., 6,000 to 15,000 rpm) to form a second capsule emulsion having second oil droplets of 0.01 to 1000 microns dispersed in the water phase (i.e., an oil-in-water emulsion). The second benefit agent can also be included in the second water phase and the resultant emulsion is a water-in-oil emulsion.

The first and second emulsions are mixed in an encapsulating reactor to obtain an emulsion mixture. A first capsule is then caused to be formed having a first capsule wall encapsulating (i.e., at least partially surrounding or fully surrounding) a first core materials (corresponding to the first oil droplet less any wall-forming materials). In the same encapsulating reactor, a second capsule is also caused to be formed having a second capsule wall encapsulating a second core materials (corresponding to the first oil droplet less any wall-forming materials). The formation of each of the first and second capsules, independently, is facilitated by raising the temperature (e.g., 40 to 150° C.), adjusting the pH (e.g., 0-7 and 7-14), adding an activation agent such as a crosslinking agent and a catalyst. By way of illustration, the temperature is kept at 20-40° C. so that a first capsule (e.g., a silica capsule) is formed. Subsequently, a crosslinking agent is added or the temperature is raised to 50-140° C. to allow the formation of the second capsule.

After the first and second capsules are formed, both are optionally cured at a predetermined temperature (e.g., 20-150° C., 20-40° C., 50-95° C., and 95-135° C.) for a predetermined period of time (e.g., 30 minutes to 48 hours, and 1-5 hours).

The first and second capsules have a different capsule wall (namely, different wall material), thickness of the capsule wall, modification of the capsule wall (e.g., presence or absence of a deposition aid, the amount of the deposition aid, and different deposition aids present on the surface of the first or second capsule wall), core material, capsule size, or combination thereof.

In some embodiments, the first emulsion and the second emulsion are prepared in one reactor. Accordingly, a first emulsion is prepared in a reactor according to the method described above. The first emulsion contains a plurality of the first oil droplets dispersed in the first water phase. In the same reactor, a second oil phase, which contains a second benefit agent and a second wall-forming material, is added to the first emulsion. Optionally, a second water phase containing a second dispersant and water is added to the reactor. The second oil phase is then emulsified in the first emulsion under high shearing at a rate of 3,000 to 20,000 rpm (e.g., 6,000 to 15,000 rpm). A second capsule emulsion is thus formed, which has a plurality of the second oil droplets dispersed in the second capsule emulsion, together with the first emulsion, in the resultant emulsion mixture. The first and second emulsions are allowed to polymerized to form a plurality of the first and second capsuled, followed by curing at a predetermined temperature (e.g., 20-150° C., 20-40° C., 50-95° C., and 95-135° C.) for a predetermined period of time (e.g., 30 minutes to 48 hours, and 1-5 hours).

In other embodiments, additional emulsions (e.g., a third, fourth, fifth, and sixth emulsions) are provided and added to the emulsion mixture to prepare additional capsules (e.g., a third, fourth, fifth, and sixth capsules). These capsules are different from each other and also the first and second capsules.

In another method of this invention, the first and second capsules are prepared in tandem. A first capsule slurry is provided. It includes a first capsule that contains a first benefit agent encapsulated by a first capsule walls formed of a first wall-forming material. The first capsule slurry can be prepared from the first emulsion described above by causing the formation of a first capsules Optionally, the first capsule slurry is cured at a first curing temperature (e.g., 20-150° C., 20-40° C., 50-95° C., 70-100° C., and 95-135° C.) for a predetermined time (e.g., 30 minutes to 48 hours, and 1-5 hours).

A second emulsion is then added to the reactor that contains the first capsule slurry. The second emulsion can be prepared in the same reactor or in a separate reactor.

To prepare the second emulsion in the same reactor that contains the first capsule slurry, a second oil phase, which contains a second benefit agent and a second wall-forming material, is added to the first capsule slurry. Optionally, a second water phase containing a second dispersant and water is added to the reactor. The second oil phase is then emulsified under high shearing at a rate of 3,000 to 20,000 rpm (e.g., 6,000 to 15,000 rpm). A second capsule emulsion is thus formed, which has a plurality of the second oil droplets to obtain a capsule mixture that contains the first capsule slurry.

To prepare the second emulsion in a separate reactor, a procedure described above can be followed. The second emulsion thus prepared is then mixed with the first capsule slurry to obtain a capsule mixture.

A second capsule is formed to give a mixed capsule slurry containing the first and second capsules. Curing the capsules yields a capsule delivery system of this invention.

The capsule walls each are formed around a benefit agent droplet by transforming a capsule wall-forming material (present in a solution containing the benefit agent) in the presence of absence of an activation agent in the same reactor. The activation agent is typically a catalyst or a cross-linker, which facilitates the formation of capsule walls.

The first and second benefit agents can be the same as long as the first and second capsules having one or more different properties, e.g., size, wall thickness, wall polymer, degree of crosslinking, and etc.

These capsule properties can be manipulated by, e.g., varying the amount of capsule wall-forming material in the emulsions, curing temperature/time, and/or droplet size, incorporating benefit agents that affect the wall properties, modulating the shear rate after each benefit agent addition, choosing the type of cross-linkers used, and any combination thereof.

When each of benefit agents is independently encapsulated, the resultant capsules can be cured at the same time or in a sequential fashion. When they are cured at the same time, a capsule containing a first benefit agent can be formed, and a second benefit agent is subsequently added, emulsified, and encapsulated. Additional benefit agents may be added and encapsulated in a similar fashion. Both the first and second capsules, along with any additional capsules, are then cured. Alternatively, the first capsule is formed and cured before a second benefit agent and wall-forming material are added to it.

Benefit agents can be encapsulated using different chemistries, e.g., different polyureas, and any combination of different polymers such as a polyurea and a polyurethane, a polyurea and a silica, a polyurethane and a melamine formaldehyde, and a starch and a polyurea. When different chemistries are used, a quenching agent is optionally added to the first capsule wall-forming reaction thereby ensuring that the second capsule wall-forming reaction does not inadvertently incorporate unreacted starting material from the first reaction. In this respect, the method of this invention also includes this optional step of adding a quenching agent to the first capsule slurry. Examples of quenching agents include, but are not limited to, an acid or base (e.g., HCl and NaOH).

Advantageously, the instant methods provide the ability to make capsules containing two or more different capsules in one batch. For example, each of the capsules can contain one or more fragrances that are different from those contained in other capsules. As another example, each of the capsules has a capsule wall that is different from those of other capsules in size, thickness, degree of cross-linking, and/or polymers contained therein. Also within the scope of this invention are methods wherein two or more batches of cured capsules are prepared separately, subsequently blended together, and then cured in one pot.

### Core-Shell Encapsulation Systems.

The capsules can be prepared following encapsulation procedures known in the art, see for example U.S. Pat. Nos. 2,800,457, 3,870,542, 3,516,941, 3,415,758, 3,041,288, 5,112,688, 6,329,057, and 6,261,483. Wall forming materials include melamine formaldehyde, polyurethane, polysiloxanes, polyurea, polyamide, polyimide, polyvinyl alcohol, polyanhydride, polyolefin, polysulfone, polysaccharide, protein, polylactide (PLA), polyglycolide (PGA), polyorthoester, polyphosphazene, silicone, lipid, modified cellulose, gums, polystyrene, and polyesters or combinations of these materials. Other polymeric materials that are functional are ethylene maleic anhydride copolymer, styrene maleic anhydride copolymer, ethylene vinyl acetate copolymer, and lactide glycolide copolymer. Biopolymers that are derived from alginate, chitosan, collagen, dextran, gelatin, and starch can also be used as the encapsulating materials. Additionally, capsules can be made via the simple or complex coacervation of gelatin. Preferred encapsulating wall polymers include those formed from isocyanates, acrylates, acrylamide, acrylate-co-acrylamide, hydrogel monomers, sol-gel precursors, gelatin, melamine-formaldehyde or urea-formaldehyde condensates, as well as similar types of aminoplasts.

### Polyurea/Polyurethane Capsules.

Polyurea capsules can be prepared using multi-functional isocyanates and multi-functional amines. See WO 2004/054362; EP 0 148149; EP 0 017 409 B1; U.S. Pat. Nos. 4,417,916, 4,124,526, 4,285,720, 4,681,806, 5,583,090, 6,340,653 6,566,306, 6,730,635, 8,299,011, WO 90/08468, and WO 92/13450.

These isocyanates contain two or more isocyanate (—NCO) groups. Suitable isocyanates include, for example, 1,5-naphthylene diisocyanate, 4,4′-diphenylmethane diisocyanate (MDI), hydrogenated MDI (H12MDI), xylylene diisocyanate (XDI), tetramethylxylol diisocyanate (TMXDI), 4,4′-diphenyldimethylmethane diisocyanate, di- and tetraalkyldiphenylmethane diisocyanate, 4,4′-dibenzyl diisocyanate, 1,3-phenylene diisocyanate, 1,4-phenylene diisocyanate, the isomers of tolylene diisocyanate (TDI), optionally in a mixture, 1-methyl-2,4-diisocyanatocyclohexane, 1,6-diisocyanato-2,2,4-trimethylhexane, 1,6-diisocyanato-2,4,4-trimethylhexane, 1-isocyanatomethyl-3-isocyanato-1,5,5-trimethylcyclohexane, chlorinated and brominated diisocyanates, phosphorus-containing diisocyanates, 4,4′-diisocyanatophenylperfluoroethane, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, phthalic acid bisisocyanatoethyl ester, also polyisocyanates with reactive halogen atoms, such as 1-chloromethylphenyl 2,4-diisocyanate, 1-bromomethylphenyl 2,6-diisocyanate, and 3,3-bischloromethyl ether 4,4′-diphenyldiisocyanate. Sulfur-containing polyisocyanates are obtained, for example, by reacting hexamethylene diisocyanate with thiodiglycol or dihydroxydihexyl sulfide. Further suitable diisocyanates are trimethylhexamethylene diisocyanate, 1,4-diisocyanatobutane, 1,2-diisocyanatododecane and dimer fatty acid diisocyanate.

The multi-functional amines contains two or more amine groups including —NH₂ and —RNH, R being substituted and unsubstituted C₁-C₂₀ alkyl, C₁-C₂₀ heteroalkyl, C₁-C₂₀ cycloalkyl, 3- to 8-membered heterocycloalkyl, aryl, and heteroaryl.

Water soluble diamines are one class of amines of use in this invention as the amine is usually present in the aqueous phase. One class of such amine is of the type:

H₂N(CH₂)ₙNH₂,

where n is ≧1. When n is 1, the amine is a diamine, ethylene diamine. When n is 2, the amine is diamine propane and so on. Exemplary amines of this type include, but are not limited to, ethylenediamine, 1,3-diaminopropane, 1,4-diaminobutane, hexanethylene diamine, hexamethylene diamine, and pentaethylenehexamine. In particular embodiments of this invention, the preferred n is 6, where the amine is a hexamethylene diamine.

Amines that have a functionality greater than 2, but less than 3 and which may provide a degree of cross linking in the shell wall are the polyalykylene polyamines of the type:

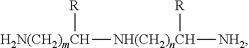

where R equals hydrogen or —CH₃, m is 1-5 and n is 1-5, e.g., diethylene triamine, triethylene tetraamine and the like. Exemplary amines of this type include, but are not limited to diethylenetriamine, bis(3-aminopropyl)amine, bis(hexamethylene)triamine.

Another class of amine that can be used in the invention is polyetheramines. They contain primary amino groups attached to the end of a polyether backbone. The polyether backbone is normally based on either propylene oxide (PO), ethylene oxide (EO), or mixed PO/EO. The ether amine can be monoamine, diamine, or triamine, based on this core structure. An example is:

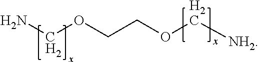

Exemplary polyetheramines include 2,2′-ethylenedioxy)bis (ethylamine) and 4,7,10-trioxa-1,13-tridecanediamine.

Other suitable amines include, but are not limited to, tris(2-aminoethyl)amine, triethylenetetramine, N,N′-bis(3-aminopropyl)-1,3-propanediamine, tetraethylene pentamine, 1,2-diaminopropane, N,N,N′,N′-tetrakis(2-hydroxyethyl)ethylene diamine, N,N,N′,N′-tetrakis(2-hydroxypropyl)ethylene diamine, branched polyethylenimine, 2,4-diamino-6-hydroxypyrimidine and 2,4,6-triaminopyrimidine.

Amphoteric amines, i.e., amines that can react as an acid as well as a base, are another class of amines of use in this invention. Examples of amphoteric amines include proteins and amino acids such as gelatin, L-lysine, L-arginine, L-lysine monohydrochloride, arginine monohydrochloride and ornithine monohydrochloride.

Guanidine amines and guanidine salts are yet another class of amines of use in this invention. Exemplary guanidine amines and guanidine salts include, but are not limited to, 1,3-diaminoguanidine monohydrochloride, 1,1-dimethylbiguanide hydrochloride, guanidine carbonate and guanidine hydrochloride.

Commercially available examples of amines include JEFFAMINE EDR-148 (where x=2), JEFFAMINE EDR-176 (where x=3) (from Huntsman). Other polyether amines include the JEFFAMINE ED Series, and JEFFAMINE TRIAMINES.

Alcohols of use as cross-linking agents typically have at least two nucleophilic centers. Exemplary alcohols include, but are not limited to, ethylene glycol, hexylene glycol, pentaerythritol, glucose, sorbitol, and 2-aminoethanol.

The preparation of polyurethane capsules can be carried out by reacting one or more of the above-referenced isocyanates with a diol or polyol in the presence of a catalyst. Diols or polyols of use in the present invention have a molecular weight in the range of 200-2000. Exemplary diols include, but are not limited to, ethylene glycol, diethylene glycol, propylene glycol, 1,4-butane diol, 1,4 hexane diol, dipropylene glycol, cyclohexyl 1,4 dimethanol, and 1,8 octane diol. Exemplary polyols include, but are not limited to, poly (ethylene glycols), poly (propylene glycols), and poly (tetramethylene glycols).

Catalysts suitable for use in the invention are amino or organometallic compounds and include, for example, 1,4-diazabicyclo[2.2.2]octane (e.g., DABCO, Air Products, Allentown, Pa.), N,N-dimethylaminoethanol, N,N-dimethylcyclohexylamine, bis-(2-dimethylaminoethyl) ether, N,N dimethylacetylamine, stannous octoate and dibutyltin dilaurate.

Table 1 below lists typical cross-liners useful for preparing the polyurea and polyurethane walls.

TABLE 1

| Active Hydrogen | | Rel Rxn Rate w/p- |
|---|---|---|
| Compound | Typical Structure | MDI @ 25° C. |
| 1° Aliphatic Amine | R—NH₂ | 100,000 |
| 2° Aliphatic Amine | R₁R₂NH | 20,000-50,000 |
| 1° Aromatic Amine | Ar—NH₂ | 200-300 |
| 1° Hydroxyl | R—CH₂—OH | 100 |
| Water | H-O-H | 100 |
| 2° Aromatic Amine | Ar—NH—R or Ar—NH—Ar′ | 100 |
| Carboxylic Acid | R—COOH | 40 |
| 2° Hydroxyl | RR′CH—OH | 30 |
| Ureas | R—NH—C(O)—NH—R′ | 15 |
| 3° Hydroxyl | R₃C—OH | 0.5 |
| Urethanes | R—NH—C(O)—O—R′ | 0.3 |
| Amide | RC(O)—NH₂ | 0.1 |

### Aminoplasts and Gelatin

A representative process used for aminoplast encapsulation is disclosed in U.S. Pat. No. 3,516,941, though it is recognized that many variations with regard to materials and process steps are possible. Another encapsulation process, i.e., gelatin encapsulation, is disclosed in U.S. Pat. No. 2,800,457. Both processes are discussed in the context of fragrance encapsulation for use in consumer products in U.S. Pat. Nos. 4,145,184 and 5,112,688 respectively. Polymer systems are well-known in the art and non-limiting examples of these include aminoplast capsules and encapsulated particles as disclosed in GB 2006709 A; the production of micro-capsules having walls comprising styrene-maleic anhydride reacted with melamine-formaldehyde precondensates as disclosed in U.S. Pat. No. 4,396,670; an acrylic acid-acrylamide copolymer, cross-linked with a melamine-formaldehyde resin as disclosed in U.S. Pat. No. 5,089,339; capsules composed of cationic melamine-formaldehyde condensates as disclosed in U.S. Pat. No. 5,401,577; melamine formaldehyde microencapsulation as disclosed in U.S. Pat. No. 3,074,845; amido-aldehyde resin in-situ polymerized capsules disclosed in EP 0 158 449 A1; etherified urea-formaldehyde polymer as disclosed in U.S. Pat. No. 5,204,185; melamine-formaldehyde microcapsules as described in U.S. Pat. No. 4,525,520; cross-linked oil-soluble melamine-formaldehyde precondensate as described in U.S. Pat. No. 5,011,634; capsule wall material formed from a complex of cationic and anionic melamine-formaldehyde precondensates that are then cross-linked as disclosed in U.S. Pat. No. 5,013,473; polymeric shells made from addition polymers such as condensation polymers, phenolic aldehydes, urea aldehydes or acrylic polymer as disclosed in U.S. Pat. No. 3,516,941; urea-formaldehyde capsules as disclosed in EP 0 443 428 A2; melamine-formaldehyde chemistry as disclosed in GB 2 062 570 A; and capsules composed of polymer or copolymer of styrene sulfonic acid in acid of salt form, and capsules cross-linked with melamine-formaldehyde as disclosed in U.S. Pat. No. 4,001,140.

### Urea-Formaldehyde and Melamine Formaldehyde Capsules

Urea-formaldehyde and melamine-formaldehyde pre-condensate capsule shell wall precursors are prepared by means of reacting urea or melamine with formaldehyde where the mole ratio of melamine or urea to formaldehyde is in the range of from about 10:1 to about 1:6, preferably from about 1:2 to about 1:5. For purposes of practicing this invention, the resulting material has a molecular weight in the range of from 156 to 3000. The resulting material may be used ‘as-is’ as a cross-linking agent for the aforementioned substituted or un-substituted acrylic acid polymer or copolymer or it may be further reacted with a C₁-C₆ alkanol, e.g., methanol, ethanol, 2-propanol, 3-propanol, 1-butanol, 1-pentanol or 1-hexanol, thereby forming a partial ether where the mole ratio of melamine/urea:formaldehyde:alkanol is in the range of 1:(0.1-6):(0.1-6). The resulting ether moiety-containing product may be used ‘as-is’ as a cross-linking agent for the aforementioned substituted or un-substituted acrylic acid polymer or copolymer, or it may be self-condensed to form dimers, trimers and/or tetramers which may also be used as cross-linking agents for the aforementioned substituted or un-substituted acrylic acid polymers or co-polymers. Methods for formation of such melamine-formaldehyde and urea-formaldehyde pre-condensates are set forth in U.S. Pat. Nos. 3,516,846 and 6,261,483, and Lee et al. (2002) J. Microencapsulation 19, 559-569.

Examples of urea-formaldehyde pre-condensates useful in the practice of this invention are URAC 180 and URAC 186, trademarks of Cytec Technology Corp. of Wilmington, Del. Examples of melamine-formaldehyde pre-condensates useful in the practice if this invention, include, but are not limited to, CYMEL U-60, CYMEL U-64 and CYMEL U-65, trademarks of Cytec Technology Corp. of Wilmington, Del. It is preferable to use, as the precondensate for cross-linking, the substituted or un-substituted acrylic acid polymer or co-polymer. In practicing this invention, the range of mole ratios of urea-formaldehyde precondensate/melamine-formaldehyde pre-condensate to substituted/un-substituted acrylic acid polymer/co-polymer is in the range of from about 9:1 to about 1:9, preferably from about 5:1 to about 1:5 and most preferably from about 2:1 to about 1:2.

In one embodiment of the invention, microcapsules with polymer(s) composed of primary and/or secondary amine reactive groups or mixtures thereof and cross-linkers can also be used. See US 2006/0248665. The amine polymers can possess primary and/or secondary amine functionalities and can be of either natural or synthetic origin. Amine-containing polymers of natural origin are typically proteins such as gelatin and albumen, as well as some polysaccharides. Synthetic amine polymers include various degrees of hydrolyzed polyvinyl formamides, polyvinylamines, polyallyl amines and other synthetic polymers with primary and secondary amine pendants. Examples of suitable amine polymers are the LUPAMIN series of polyvinyl formamides available from BASF. The molecular weights of these materials can range from 10,000 to 1,000,000.

Urea-formaldehyde or melamine-formaldehyde capsules can also include formaldehyde scavengers, which are capable of binding free formaldehyde. When the capsules are for use in aqueous media, formaldehyde scavengers such as sodium sulfite, melamine, glycine, and carbohydrazine are suitable. When the capsules are aimed to be used in products having low pH, e.g., fabric care conditioners, formaldehyde scavengers are preferably selected from beta diketones, such as beta-ketoesters, or from 1,3-diols, such as propylene glycol. Preferred beta-ketoesters include alkyl-malonates, alkyl aceto acetates and polyvinyl alcohol aceto acetates.

As indicated, the capsules of this invention can be prepared by conventional methods to encapsulate fragrances. In some embodiments, the fragrance is encapsulated by a polymer in the presence of a capsule formation aid, e.g., a surfactant or dispersant. Classes of protective colloid or emulsifier of use as surfactants or dispersants include maleic-vinyl copolymers such as the copolymers of vinyl ethers with maleic anhydride or acid, sodium lignosulfonates, maleic anhydride/styrene copolymers, ethylene/maleic anhydride copolymers, and copolymers of propylene oxide, ethylenediamine and ethylene oxide, polyvinylpyrrolidone, polyvinyl alcohols, carboxymethyl cellulose, fatty acid esters of polyoxyethylenated sorbitol and sodium dodecylsulfate.

Commercially available surfactants include, but are not limited to, sulfonated naphthalene-formaldehyde condensates such as MORWET D425 (Akzo Nobel); partially hydrolyzed polyvinyl alcohols such as MOWIOLs, e.g., MOWIOL 3-83 (Air Products); ethylene oxide-propylene oxide block copolymers or poloxamers such as PLURONIC, SYNPERONIC or PLURACARE materials (BASF); sulfonated polystyrenes such as FLEXAN II (Akzo Nobel); and ethylene-maleic anhydride polymers such as ZEMAC (Vertellus Specialties Inc.)

Typically, hydrocolloids or adjuvants are used to improve the colloidal stability of the capsule suspension or slurry against coagulation, sedimentation and creaming. As such, such processing aids can also be used in conjunction with the microcapsules of this invention. As used herein, the term “hydrocolloid” refers to a broad class of water-soluble or water-dispersible polymers having anionic, cationic, zwitterionic or nonionic character. In particular embodiments, the capsule suspension includes a nonionic polymer, cationic polymer, anionic polymer, anionic surfactant, or a combination thereof. In certain embodiments, the nonionic polymer is a polyvinylpyrrolidone (PVP), polyvinyl alcohol (PVA) polyethylene glycol (PEG), Polyethylene oxide (PEO), or polyethylene oxide-polypropylene oxide (PEO-PPO), polyethylene oxide-polypropylene oxide-polyethylene oxide (PEO-PPO-PEO). In other embodiments, the cationic polymer is Polyquaterium-6 (polydiallyldimethylammonium chloride), Polyquaternium-11 (vinyl pyrrolidone/dimethylaminoethyl methacrylate copolymer) or Polyquaternium-47 (acrylic acid/methacrylamidopropyl trimethyl ammonium chloride/methyl acrylate terpolymer). In yet other embodiments, the anionic polymer is a polystyrene sulfonic acid, polyacrylic acid, hyaluronic acid, sodium alginate, or sodium carboxymethylcellulose (CMC). In still other embodiments, the anionic surfactant is sodium laureth sulfate (SLS) or a complex ester of phosphoric acid and ethoxylated cosmetic grade oleyl alcohol (e.g., CRODAFOS 010A-SS-(RB)).

Other hydrocolloids useful in the present invention include polycarbohydrates, such as starch, modified starch, dextrin, maltodextrin, and cellulose derivatives, and their quaternized forms; natural gums such as alginate esters, carrageenan, xanthanes, agar-agar, pectins, pectic acid, and natural gums such as gum arabic, gum tragacanth and gum karaya, guar gums and quaternized guar gums; gelatin, protein hydrolysates and their quaternized forms; synthetic polymers and copolymers, such as poly(vinyl pyrrolidone-co-vinyl acetate), poly(vinyl alcohol-co-vinyl acetate), poly((met)acrylic acid), poly(maleic acid), poly(alkyl(meth)acrylate-co-(meth)acrylic acid), poly(acrylic acid-co-maleic acid)copolymer, poly(alkyleneoxide), poly(vinylmethylether), poly(vinylether-co-maleic anhydride), and the like, as well as poly-(ethyleneimine), poly((meth)acrylamide), poly(alkyleneoxide-co-dimethylsiloxane), poly(amino dimethylsiloxane), and their quartenized forms.

The capsule formation aid may also be used in combination with carboxymethyl cellulose and/or a surfactant during processing to facilitate capsule formation. Examples of surfactants that can be used in combination with the capsule formation aid include, but are not limited to, cetyl trimethyl ammonium chloride (CTAC), poloxamers such as PLURONICS (e.g., PLURONIC F127), PLURAFAC (e.g., PLURAFAC F127), or MIRANET-N, saponins such as QNATURALE (National Starch Food Innovation); or a gum Arabic such as Seyal or Senegal. The amount of surfactant present in the capsule slurry can vary depending on the surfactant used. In some embodiments the amount of surfactant is in the range of 0.05 to 0.2 weight percent, in particular when CTAC is employed. In another embodiment, the amount of surfactant is in the range of 1 to 3 weight percent when a saponin or gum arabic is used.

When combined with carboxymethyl cellulose (also referred to as CMC), the lighter color polyvinyl alcohol, carboxymethyl cellulose is preferred. In certain embodiments, the carboxymethyl cellulose polymer has a molecular weight range between about 90,000 Daltons to 1,500,000 Daltons, more preferably between about 250,000 Daltons to 750,000 Daltons and most preferably between 400,000 Daltons to 750,000 Daltons. The carboxymethyl cellulose polymer has a degree of substitution between about 0.1 to about 3, more preferably between about 0.65 to about 1.4, and most preferably between about 0.8 to about 1.0.

The carboxymethyl cellulose polymer is present in the capsule slurry at a level from about 0.1 weight percent to about 2 weight percent and more preferably from about 0.3 weight percent to about 0.7 weight percent.

In some embodiments, CMC-modified microcapsules may provide a perceived fragrance intensity increase of greater than about 15%, and more preferably an increase of greater than about 25% as compared to microcapsules not including CMC.

The diameter of the capsules produced in accordance with this invention can vary from about 10 nanometers to about 1000 microns, preferably from about 50 nanometers to about 150 microns and is most preferably from about 2 to about 15 microns. The capsule distribution can be narrow, broad, or multi-modal. In particular embodiments, the delivery system of the invention has a multi-modal distribution indicative of different types of capsule chemistries.

In some embodiments, the capsule suspension prepared in accordance with the present invention is subsequently purified. Purification can be achieved by washing the capsule slurry with water, e.g., deionized or double deionized water, until a neutral pH is achieved. For the purposes of the present invention, the capsule suspension can be washed using any conventional method including the use of a separatory funnel, filter paper, centrifugation and the like. The capsule suspension can be washed one, two, three, four, five, six, seven, eight, nine, ten or more times until a neutral pH, i.e., pH 7±0.5, is achieved. The pH of the purified capsules can be determined using any conventional method including, but not limited to pH paper, pH indicators, or a pH meter.

A capsule suspension of this invention is “purified” in that it is 80%, 90%, 95%, 97%, 98% or 99% homogeneous to capsules. In accordance with the present invention, purity is achieved by washing the capsules until a neutral pH is achieved, which is indicative of removal of unwanted impurities and/or starting materials, e.g., polyisocyanate, cross-linking agent and the like.

In certain embodiments of this invention, the purification of the capsules includes the additional step of adding a salt to the capsule suspension prior to the step of washing the capsule suspension with water. Exemplary salts of use in this step of the invention include, but are not limited to, sodium chloride, potassium chloride or bi-sulphite salts.

The delivery system of this invention can be a slurry or in a solid form. When used as a slurry, the delivery system can be sprayed onto a consumer product, e.g., a fabric care product. By way of illustration, a liquid delivery system containing different types of capsules is sprayed onto a detergent powder during blending to make granules. See US 2011/0190191. In order to increase fragrance load, water-absorbing material, such as zeolite, can be added to the delivery system.

Alternatively, granulates in a consumer product are prepared in a mechanical granulator in the presence of a granulation auxiliary such as non-acid water-soluble organic crystalline solids. See WO 2005/097962.

The delivery system can also be spray dried to a solid form. The solid delivery system is then incorporated into a consumer product.

Benefit Agents.

Benefit agents include, but are not limited to, flavors and fragrances. Suitable fragrances include without limitation, any combination of fragrance oil, essential oil, plant extract or mixture thereof that is compatible with, and capable of being encapsulated by a polymer. Individual perfume ingredients that can be included in the capsules of this invention include fragrances containing:

i) hydrocarbons, such as, for example, 3-carene, α-pinene, β-pinene, α-terpinene, γ-terpinene, p-cymene, bisabolene, camphene, caryophyllene, cedrene, farnesene, limonene, longifolene, myrcene, ocimene, valencene, (E,Z)-1,3,5-undecatriene, styrene, and diphenylmethane;

ii) aliphatic alcohols, such as, for example, hexanol, octanol, 3-octanol, 2,6-dimethylheptanol, 2-methyl-2-heptanol, 2-methyl-2-octanol, (E)-2-hexenol, (E)- and (Z)-3-hexenol, 1-octen-3-ol, a mixture of 3,4,5,6,6-pentamethyl-3/4-hepten-2-ol and 3,5,6,6-tetramethyl-4-methyleneheptan-2-ol, (E,Z)-2,6-nonadienol, 3,7-dimethyl-7-methoxyoctan-2-ol, 9-decenol, 10-undecenol, 4-methyl-3-decen-5-ol, aliphatic aldehydes and their acetals such as for example hexanal, heptanal, octanal, nonanal, decanal, undecanal, dodecanal, tridecanal, 2-methyloctanal, 2-methylnonanal, (E)-2-hexenal, (Z)-4-heptenal, 2,6-dimethyl-5-heptenal, 10-undecenal, (E)-4-decenal, 2-dodecenal, 2,6,10-trimethyl-5,9-undecadienal, heptanal-diethylacetal, 1,1-dimethoxy-2,2,5-trimethyl-4-hexene, and citronellyl oxyacetaldehyde;

iii) aliphatic ketones and oximes thereof, such as, for example, 2-heptanone, 2-octanone, 3-octanone, 2-nonanone, 5-methyl-3-heptanone, 5-methyl-3-heptanone oxime, 2,4,4,7-tetramethyl-6-octen-3-one, aliphatic sulfur-containing compounds, such as for example 3-methylthiohexanol, 3-methylthiohexyl acetate, 3-mercaptohexanol, 3-mercaptohexyl acetate, 3-mercaptohexyl butyrate, 3-acetylthiohexyl acetate, 1-menthene-8-thiol, and aliphatic nitriles (e.g., 2-nonenenitrile, 2-tridecenenitrile, 2,12-tridecenenitrile, 3,7-dimethyl-2,6-octadienenitrile, and 3,7-dimethyl-6-octenenitrile);

iv) aliphatic carboxylic acids and esters thereof, such as, for example, (E)- and (Z)-3-hexenylformate, ethyl acetoacetate, isoamyl acetate, hexyl acetate, 3,5,5-trimethylhexyl acetate, 3-methyl-2-butenyl acetate, (E)-2-hexenyl acetate, (E)- and (Z)-3-hexenyl acetate, octyl acetate, 3-octyl acetate, 1-octen-3-yl acetate, ethyl butyrate, butyl butyrate, isoamyl butyrate, hexylbutyrate, (E)- and (Z)-3-hexenyl isobutyrate, hexyl crotonate, ethylisovalerate, ethyl-2-methyl pentanoate, ethyl hexanoate, allyl hexanoate, ethyl heptanoate, allyl heptanoate, ethyl octanoate, ethyl-(E,Z)-2,4-decadienoate, methyl-2-octinate, methyl-2-noninate, allyl-2-isoamyl oxyacetate, and methyl-3,7-dimethyl-2,6-octadienoate;

v) acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5,7-octatrien-1-ol; as well as formates, acetates, propionates, isobutyrates, butyrates, isovalerates, pentanoates, hexanoates, crotonates, tiglinates and 3-methyl-2-butenoates thereof;

vi) acyclic terpene aldehydes and ketones, such as, for example, geranial, neral, citronellal, 7-hydroxy-3,7-dimethyloctanal, 7-methoxy-3,7-dimethyloctanal, 2,6,10-trimethyl-9-undecenal, α-sinensal, β-sinensal, geranylacetone, as well as the dimethyl- and diethylacetals of geranial, neral and 7-hydroxy-3,7-dimethyloctanal;

vii) cyclic terpene alcohols, such as, for example, menthol, isopulegol, alpha-terpineol, terpinen-4-ol, menthan-8-ol, menthan-1-ol, menthan-7-ol, borneol, isoborneol, linalool oxide, nopol, cedrol, ambrinol, vetiverol, guaiol, and the formates, acetates, propionates, isobutyrates, butyrates, isovalerates, pentanoates, hexanoates, crotonates, tiglinates and 3-methyl-2-butenoates of alpha-terpineol, terpinen-4-ol, methan-8-ol, methan-1-ol, methan-7-ol, borneol, isoborneol, linalool oxide, nopol, cedrol, ambrinol, vetiverol, and guaiol;

viii) cyclic terpene aldehydes and ketones, such as, for example, menthone, isomenthone, 8-mercaptomenthan-3-one, carvone, camphor, fenchone, α-ionone, β-ionone, α-n-methylionone, β-n-methylionone, α-isomethylionone, β-isomethylionone, alpha-irone, α-damascone, β-damascone, β-damascenone, δ-damascone, γ-damascone, 1-(2,4,4-trimethyl-2-cyclohexen-1-yl)-2-buten-1-one, 1,3,4,6,7,8a-hexahydro-1,1,5,5-tetramethyl-2H-2,4a-methanonaphthalen-8(5H-)-one, nootkatone, dihydronootkatone; acetylated cedarwood oil (cedryl methyl ketone);

ix) cyclic alcohols, such as, for example, 4-tert-butylcyclohexanol, 3,3,5-trimethylcyclohexanol, 3-isocamphylcyclohexanol, 2,6,9-trimethyl-Z2,Z5,E9-cyclododecatrien-1-ol, 2-isobutyl-4-methyltetrahydro-2H-pyran-4-ol;

x) cycloaliphatic alcohols, such as, for example, alpha, 3,3-trimethylcyclo-hexylmethanol, 2-methyl-4-(2,2,3-trimethyl-3-cyclopent-1-yl)butanol, 2-methyl-4-(2,2,3-trimethyl-3-cyclopent-1-yl)-2-buten-1-ol, 2-ethyl-4-(2,2,3-trimethyl-3-cyclopent-1-yl)-2-buten-1-ol, 3-methyl-5-(2,2,3-trimethyl-3-cyclopent-1-yl)-pentan-2-ol, 3-methyl-5-(2,2,3-trimethyl-3-cyclopent-1-yl)-4-penten-2-ol, 3,3-dimethyl-5-(2,2,3-trimethyl-3-cyclopent-1-yl)-4-penten-2-ol, 1-(2,2,6-trimethylcyclohexyl)pentan-3-ol, 1-(2,2,6-trimethylcyclohexyl)hexan-3-ol;

xi) cyclic and cycloaliphatic ethers, such as, for example, cineole, cedryl methyl ether, cyclododecyl methyl ether;

xii) (ethoxymethoxy)cyclododecane; alpha-cedrene epoxide, 3a,6,6,9a-tetramethyldodecahydronaphtho[2,1-b]furan, 3a-ethyl-6,6,9a-trimethyldodecahydronaphtho[2,1-b]furan, 1,5,9-trimethyl-13-oxabicyclo[10.1.0]-trideca-4,8-diene, rose oxide, 2-(2,4-dimethyl-3-cyclohexen-1-yl)-5-methyl-5-(1-methylpropyl)-1,3-dioxan;

xiii) cyclic ketones, such as, for example, 4-tert-butylcyclohexanone, 2,2,5-trimethyl-5-pentylcyclopentanone, 2-heptylcyclopentanone, 2-pentylcyclopentanone, 2-hydroxy-3-methyl-2-cyclopenten-1-one, 3-methyl-cis-2-penten-1-yl-2-cyclopenten-1-one, 3-methyl-2-pentyl-2-cyclopenten-1-one, 3-methyl-4-cyclopentadecenone, 3-methyl-1-cyclopentadecenone, 3-methylcyclopentadecanone, 4-(1-ethoxyvinyl)-3,3,5,5-tetramethylcyclohexanone, 4-tert-pentylcyclohexanone, 5-cyclohexadecen-1-one, 6,7-dihydro-1,1,2,3,3-pentamethyl-4(5H)-indanone, 5-cyclohexadecen-1-one, 8-cyclohexadecen-1-one, 9-cycloheptadecen-1-one, cyclopentadecanone, cycloaliphatic aldehydes, such as, for example, 2,4-dimethyl-3-cyclohexene carbaldehyde, 2-methyl-4-(2,2,6-trimethyl-cyclohexen-1-yl)-2-butenal, 4-(4-hydroxy-4-methylpentyl)-3-cyclohexene carbaldehyde, 4-(4-methyl-3-penten-1-yl)-3-cyclohexene carbaldehyde;

xiv) cycloaliphatic ketones, such as, for example, 1-(3,3-dimethylcyclohexyl)-4-penten-1-one, 1-(5,5-dimethyl-1-cyclohexen-1-yl)-4-penten-1-one, 2,3,8,8-tetramethyl-1,2,3,4,5,6,7,8-octahydro-2-naphtalenyl methyl-ketone, methyl-2,6,10-trimethyl-2,5,9-cyclododecatrienyl ketone, tert-butyl-(2,4-dimethyl-3-cyclohexen-1-yl)ketone;

xv) esters of cyclic alcohols, such as, for example, 2-tert-butylcyclohexyl acetate, 4-tert-butylcyclohexyl acetate, 2-tert-pentylcyclohexyl acetate, 4-tert-pentylcyclohexyl acetate, decahydro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl-isobutyrate, 4,7-methanooctahydro-5 or 6-indenyl acetate;

xvi) esters of cycloaliphatic carboxylic acids, such as, for example, allyl 3-cyclohexyl-propionate, allyl cyclohexyl oxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cyclohexenecarboxylate, ethyl 2-methyl-1,3-dioxolane-2-acetate;

xvii) aromatic and aliphatic alcohols, such as, for example, benzyl alcohol, 1-phenylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl)propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl)ethanol;

xviii) esters of aliphatic alcohols and aliphatic carboxylic acids, such as, for example, benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, α-trichloromethylbenzyl acetate, α,α-dimethylphenylethyl acetate, alpha, alpha-dimethylphenylethyl butyrate, cinnamyl acetate, 2-phenoxyethyl isobutyrate, 4-methoxybenzyl acetate, araliphatic ethers, such as for example 2-phenylethyl methyl ether, 2-phenylethyl isoamyl ether, 2-phenylethyl-1-ethoxyethyl ether, phenylacetaldehyde dimethyl acetal, phenylacetaldehyde diethyl acetal, hydratropaaldehyde dimethyl acetal, phenylacetaldehyde glycerol acetal, 2,4,6-trimethyl-4-phenyl-1,3-dioxane, 4,4a,5,9b-tetrahydroindeno[1,2-d]-m-dioxin, 4,4a,5,9b-tetrahydro-2,4-dimethylindeno[1,2-d]-m-dioxin;

xix) aromatic and aliphatic aldehydes, such as, for example, benzaldehyde; phenylacetaldehyde, 3-phenylpropanal, hydratropaldehyde, 4-methylbenzaldehyde, 4-methylphenylacetaldehyde, 3-(4-ethylphenyl)-2,2-dimethylpropanal, 2-methyl-3-(4-isopropylphenyl)propanal, 2-methyl-3-(4-tert-butylphenyl)propanal, 3-(4-tert-butylphenyl)propanal, cinnamaldehyde, alpha-butylcinnamaldehyde, alpha-amylcinnamaldehyde, alpha-hexylcinnamaldehyde, 3-methyl-5-phenylpentanal, 4-methoxybenzaldehyde, 4-hydroxy-3-methoxybenzaldehyde, 4-hydroxy-3-ethoxybenzaldehyde, 3,4-methylene-dioxybenzaldehyde, 3,4-dimethoxybenzaldehyde, 2-methyl-3-(4-methoxyphenyl)propanal, 2-methyl-3-(4-methylendioxyphenyl)propanal;

xx) aromatic and aliphatic ketones, such as, for example, acetophenone, 4-methylacetophenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl)ethanone, benzophenone, 1,1,2,3,3,6-hexamethyl-5-indanyl methyl ketone, 6-tert-butyl-1,1-dimethyl-4-indanyl methyl ketone, 1-[2,3-dihydro-1,1,2,6-tetramethyl-3-(1-methyl-ethyl)-1H-5-indenyl]ethanone, 5′,6′,7′,8′-tetrahydro-3′,5′,5′,6′,8′,8′-hexamethyl-2-acetonaphthone;

xxi) aromatic and araliphatic carboxylic acids and esters thereof, such as, for example, benzoic acid, phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3,6-dimethylbenzoate, ethyl 3-phenylglycidate, ethyl 3-methyl-3-phenylglycidate;

xxii) nitrogen-containing aromatic compounds, such as, for example, 2,4,6-trinitro-1,3-dimethyl-5-tert-butylbenzene, 3,5-dinitro-2,6-dimethyl-4-tert-butylacetophenone, cinnamonitrile, 5-phenyl-3-methyl-2-pentenonitrile, 5-phenyl-3-methylpentanonitrile, methyl anthranilate, methy-N-methylanthranilate, Schiff's bases of methyl anthranilate with 7-hydroxy-3,7-dimethyloctanal, 2-methyl-3-(4-tert-butylphenyl)propanal or 2,4-dimethyl-3-cyclohexene carbaldehyde, 6-isopropylquinoline, 6-isobutylquinoline, 6-sec-butylquinoline, indole, skatole, 2-methoxy-3-isopropylpyrazine, 2-isobutyl-3-methoxypyrazine;

xxiii) phenols, phenyl ethers and phenyl esters, such as, for example, estragole, anethole, eugenol, eugenyl methyl ether, isoeugenol, isoeugenol methyl ether, thymol, carvacrol, diphenyl ether, beta-naphthyl methyl ether, beta-naphthyl ethyl ether, beta-naphthyl isobutyl ether, 1,4-dimethoxybenzene, eugenyl acetate, 2-methoxy-4-methylphenol, 2-ethoxy-5-(1-propenyl)phenol, p-cresyl phenylacetate;

xxiv) heterocyclic compounds, such as, for example, 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one;

xxv) lactones, such as, for example, 1,4-octanolide, 3-methyl-1,4-octanolide, 1,4-nonanolide, 1,4-decanolide, 8-decen-1,4-olide, 1,4-undecanolide, 1,4-dodecanolide, 1,5-decanolide, 1,5-dodecanolide, 1,15-pentadecanolide, cis- and trans-11-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene-1,12-dodecanedioate, ethylene-1,13-tridecanedioate, coumarin, 2,3-dihydrocoumarin, and octahydrocoumarin; and

xxvi) essential oils, concretes, absolutes, resins, resinoids, balsams, tinctures such as for example ambergris tincture, amyris oil, angelica seed oil, angelica root oil, aniseed oil, valerian oil, basil oil, tree moss absolute, bay oil, armoise oil, benzoe resinoid, bergamot oil, beeswax absolute, birch tar oil, bitter almond oil, savory oil, buchu leaf oil, cabreuva oil, cade oil, calamus oil, camphor oil, cananga oil, cardamom oil, cascarilla oil, cassia oil, cassie absolute, castoreum absolute, cedar leaf oil, cedar wood oil, cistus oil, citronella oil, lemon oil, copaiba balsam, copaiba balsam oil, coriander oil, costus root oil, cumin oil, cypress oil, davana oil, dill weed oil, dill seed oil, eau de brouts absolute, oakmoss absolute, elemi oil, estragon oil, eucalyptus citriodora oil, eucalyptus oil (cineole type), fennel oil, fir needle oil, galbanum oil, galbanum resin, geranium oil, grapefruit oil, guaiacwood oil, gurjun balsam, gurjun balsam oil, helichrysum absolute, helichrysum oil, ginger oil, iris root absolute, iris root oil, jasmine absolute, calamus oil, blue camomile oil, Roman camomile oil, carrot seed oil, cascarilla oil, pine needle oil, spearmint oil, caraway oil, labdanum oil, labdanum absolute, labdanum resin, lavandin absolute, lavandin oil, lavender absolute, lavender oil, lemon-grass oil, lovage oil, lime oil distilled, lime oil expressed, linaloe oil, Litsea cubeba oil, laurel leaf oil, mace oil, marjoram oil, mandarin oil, massoi (bark) oil, mimosa absolute, ambrette seed oil, musk tincture, clary sage oil, nutmeg oil, myrrh absolute, myrrh oil, myrtle oil, clove leaf oil, clove bud oil, neroli oil, olibanum absolute, olibanum oil, opopanax oil, orange flower absolute, orange oil, origanum oil, palmarosa oil, patchouli oil, perilla oil, Peru balsam oil, parsley leaf oil, parsley seed oil, petitgrain oil, peppermint oil, pepper oil, pimento oil, pine oil, pennyroyal oil, rose absolute, rosewood oil, rose oil, rosemary oil, Dalmatian sage oil, Spanish sage oil, sandal-wood oil, celery seed oil: spike-lavender oil, star anise oil, storax oil, tagetes oil, fir needle oil, tea tree oil, turpentine oil, thyme oil, Tolu balsam, tonka bean absolute, tuberose absolute, vanilla extract, violet leaf absolute, verbena oil, vetiver oil, juniperberry oil, wine lees oil, wormwood oil, wintergreen oil, ylang-ylang oil, hyssop oil, civet absolute, cinnamon leaf oil, cinnamon bark oil, and fractions thereof or ingredients isolated therefrom.

Other than fragrances, flavors can also be used as benefit agents. Suitable flavors include essential oils, fractions thereof, or individual aroma substances. Nonlimiting examples are: extracts from natural raw materials, such as essential oils, concretes, absolutes, resins, resinoids, balsams and tinctures, such as aniseed oil; basil oil; bergamot oil; bitter almond oil; camphor oil; lemon oil; eucalyptus oil; geranium oil; grapefruit oil; ginger oil; camomile oil; spearmint oil, caraway oil, lime oil; mandarin oil; clove (blossom) oil, orange oil; peppermint oil; rose oil; rosemary oil; sage oil; yarrow oil; star aniseed oil; thyme oil; vanilla extract; juniper berry oil; wintergreen oil; cinnamon leaf oil; cinnamon bark oil; and fractions thereof and constituents isolated therefrom. More exemplary flavors include anethol, menthol, 1-menthol, menthone, isomenthone, menthyl acetate, menthofuran, menthyl methylether, mintlactone, eucalyptol, limonene, eugenol, alpha-pinene, beta-pinene, cis-sabinene hydrate, 3-octanol, 1-carvone, gamma-octalactone, gamma-nonalactone, germacrene-D, viridiflorol, 1,3E,5Z-undecatriene, isopulegol, piperitone, 2-butanone, ethyl formiate, 3-octyl acetate, isoamyl isovalerianate, hexanol, hexanal, cis-3-hexenol, linalool, alpha-terpineol, cis-/trans-carvyl acetate, p-cymol, thymol, 4,8-dimethyl-3,7-nonadien-2-one, damascenone, damascone, rose oxide, dimethyl sulfide, fenchol, acetaldehyde diethylacetal, cis-4-heptenal, isobutyraldehyde, isovaleraldehyde, cis-jasmone, anisaldehyde, methyl salicylate, myrtenyl acetate, 8-ocimenyl acetate, 2-phenylethyl alcohol, 2-phenylethyl isobutyrate, 2-phenylethyl isovalerate, cinnamic aldehyde, geraniol and nerol.

Flavor oils may contain the following solvents/diluents: ethanol, vegetable oil triglycerides, 1,2-propylene glycol, benzyl alcohol, triacetin (glycerol triacetate), diacetin (glycerol diacetate), triethyl citrate, glycerol.

In some embodiments, the amount of encapsulated fragrance or flavor is from about 0.5% to about 80% of the total capsule suspension or capsule slurry, preferably from about 5% to about 60% of the total capsule suspension or capsule slurry, and most preferably from about 20% to about 50% of the total capsule suspension or capsule slurry. In some embodiments, each encapsulated fragrance or flavor is an equal proportion of the total encapsulated fragrance oil. By way of illustration, in encapsulation system containing a first and second fragrances, each of the first and second fragrances is 50% of the total encapsulated fragrances. Likewise, in encapsulation system containing a first, second and third fragrances, each of the first, second and third fragrances is ˜33% by weight of the total encapsulated fragrances. In other embodiments of this invention, the amount of fragrances in the first and second capsules can be tailored to provide benefits to consumers at various moments. By way of illustration, the first fragrance is 20 to 80% by weight of the total encapsulated fragrances and the second fragrance is 80 to 20%.

In addition to the fragrances and flavors, the present invention contemplates the incorporation of solvent materials into one or more of the capsules. The solvent materials are hydrophobic materials that are miscible with the fragrances or flavors. The solvent materials serve to increase the compatibility of various benefit agents, increase the overall hydrophobicity of the mixture containing the benefit agents, influence the vapor pressure, or serve to structure the mixture. Suitable solvents are those having reasonable affinity for the benefit agents and a C log P greater than 2.5, preferably greater than 3.5 and more preferably greater than 5.5. In some embodiments, the solvent is combined with the benefit agents that have C log P values as set forth above. It should be noted that selecting a solvent and benefit agent with high affinity for each other will result in improvement in stability. Suitable solvents include triglyceride oil, mono and diglycerides, mineral oil, silicone oil, diethyl phthalate, polyalpha olefins, castor oil, isopropyl myristate, mono-, di- and tri-esters and mixtures thereof, fatty acids, and glycerine. The fatty acid chain can range from C₄-C₂₆ and can have any level of unsaturation. For instance, one of the following solvents can be used: capric/caprylic triglyceride known as NEOBEE M5 (Stepan Corporation); the CAPMUL series by Abitec Corporation (e.g., CAPMUL MCM); isopropyl myristate; fatty acid esters of polyglycerol oligomers, e.g., R2CO—[OCH₂—CH(OCOR1)-CH₂O—]ₙ, where R1 and R2 can be H or C₄-C₂₆ aliphatic chains, or mixtures thereof, and n ranges between 2 and 50, preferably 2 and 30; nonionic fatty alcohol alkoxylates like the NEODOL surfactants by BASF; the dobanol surfactants by Shell Corporation or the BIO-SOFT surfactants by Stepan, wherein the alkoxy group is ethoxy, propoxy, butoxy, or mixtures thereof and said surfactants can be end-capped with methyl groups in order to increase their hydrophobicity; di- and tri-fatty acid chain containing nonionic, anionic and cationic surfactants, and mixtures thereof; fatty acid esters of polyethylene glycol, polypropylene glycol, and polybutylene glycol, or mixtures thereof; polyalphaolefins such as the EXXONMOBIL PURESYM PAO line; esters such as the EXXONMOBIL PURESYN esters; mineral oil; silicone oils such polydimethyl siloxane and polydimethylcyclosiloxane; diethyl phthalate; di-octyl adipate and di-isodecyl adipate.

While no solvent is needed in the core, it is preferable that the level of solvent in the core of the microcapsule product is about 80 wt % or less, preferably about 50 wt % or less (e.g., 0-20 wt %).

When the benefit agent is a fragrance, it is preferred that fragrance ingredients within a fragrance having a high C log P are employed. For instance, the ingredients having a C log P value between 2 and 7 (e.g., between 2 and 6, and between 2 and 5) are about 25% or greater (e.g., 50% or greater and 90% or greater) by the weight of the fragrance. Those skilled in the art will appreciate that many fragrances can be created employing various solvents and fragrance ingredients. The use of relatively low to intermediate C log P fragrance ingredients will result in fragrances that are suitable for encapsulation. These fragrances are generally water-insoluble, to be delivered through the capsule systems of this invention onto consumer products in different stages such as damp and dry fabric. Without encapsulation, the free fragrances would normally have evaporated or dissolved in water during use, e.g., wash. Whilst high log P materials have excellent encapsulation properties they are generally well delivered from a regular (non-encapsulated) fragrance in a consumer product. Such fragrance chemicals would generally only need encapsulation for overall fragrance character purposes, very long-lasting fragrance delivery, or overcoming incompatibility with the consumer product, e.g., fragrance materials that would otherwise be instable, cause thickening or discoloration of the product or otherwise negatively affect desired consumer product properties.

The benefit agent to be encapsulated can be dispersed in aqueous solutions in the absence/presence of polymers, pre-condensates, surfactants, scavengers and the like prior to formation of capsules.

When formaldehyde is used, e.g., in preparing melamine formaldehyde capsules, the delivery system may include formaldehyde scavengers such as those described in US Patent Application Publications 2007/0138674 and 2009/0258042.

Other than scavengers, one or more adjunct material may be added to the delivery system in the amount of from about 0.01 weight % to about 25 weight % (e.g., from about 0.5 weight % to about 10 weight %).

The adjunct material can be a solubility modifier, an antibacterial, a sunscreen active, an antioxidant, a malodor counteracting agent, a density modifier, a stabilizer, a viscosity modifier, a pH modifier, or any combination thereof. These modifiers can be present in the wall or core of the capsules, or outside the capsules in the delivery system of this invention. Preferably, they are in the core as a core modifier.

Nonlimiting examples of a solubility modifier include surfactants (e.g., SLS and Tween 80), acidic compounds (e.g., mineral acids such as sulfuric acid, hydrochloric acid, nitric acid, and phosphoric acid, and carboxylic acids such as acetic acid, citric acid, gluconic acid, glucoheptonic acid, and lactic acid), basic compounds (e.g., ammonia, alkali metal and alkaline earth metal hydroxides, primary, secondary, or tertiary amines, and primary, secondary, or tertiary alkanolamines), ethyl alcohol, glycerol, glucose, galactose, inositol, mannitol, glactitol, adonitol, arabitol, and amino acids.

Exemplary antibacterials include bisguanidines (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol), triclosan (i.e., 2, 4, 4′-trichloro-2′hydroxy-diphenyl ether), thymol, and triclocarban.

Suitable sunscreen actives include oxybenzone, octylmethoxy cinnamate, butylmethoxy dibenzoyln ethane, p-aminobenzoic acid and octyl dimethyl-p-aminobenzoic acid.

Examples of antioxidants include beta-carotene, vitamin C (Ascorbic Acid) or an ester thereof, vitamin A or an ester thereof, vitamin E or an ester thereof, lutein or an ester thereof, lignan, lycopene, selenium, flavonoids, vitamin-like antioxidants such as coenzyme Q10 (CoQ10) and glutathione, and antioxidant enzymes such as superoxide dismutase (SOD), catalase, and glutathione peroxidase.

Malodor counteracting agents include, but not limited to, an α,β-unsaturated carbonyl compounds including but not limited to those disclosed in U.S. Pat. No. 6,610,648 and EP 2,524,704, amyl cinnamaldehyde, benzophenone, benzyl benzoate, benzyl isoeugenol, benzyl phenyl acetate, benzyl salicylate, butyl cinnamate, cinnamyl butyrate, cinnamyl isovalerate, cinnamyl propionate, decyl acetate, ethyl myristate, isobutyl cinnamate, isoamyl salicylate, phenethyl benzoate, phenethyl phenyl acetate, triethyl citrate, tripropylene glycol n-butyl ether, isomers of bicyclo[2.2.1]hept-5-ene-2-carboxylic acid, ethyl ester, nano silver, and zinc undecenylate. More suitable malodor counteracting agents can be found in US 2013/0101544 and 2013/0101545.

The density of the capsule slurry and/or the oil core can be adjusted so that the capsule compositions have a substantially uniform distribution of the capsules using known density modifiers or technologies such as those described in Patent Application Publications WO 2000/059616, EP 1 502 646, and EP 2 204 155. Suitable density modifiers include hydrophobic materials and materials having a desired molecular weight (e.g., higher than about 12,000), such as silicone oils, petrolatums, vegetable oils, especially sunflower oil and rapeseed oil, and hydrophobic solvents having a desired density (e.g., less than about 1,000 Kg/m³ at 25° C., such as limonene and octane.

In some embodiments, a stabilizer (e.g., a colloidal stabilizer) is added to capsule compositions to stabilize the emulsion and/or capsule slurry. Examples of colloidal stabilizers are polyvinyl alcohol, cellulose derivatives such hydroxyethyl cellulose, polyethylene oxide, copolymers of polyethylene oxide and polyethylene or polypropylene oxide, or copolymers of acrylamide and acrylic acid.

Viscosity control agents (e.g., suspending agents), which may be polymeric or colloidal (e.g., modified cellulose polymers such as methylcellulose, hydoxyethylcellulose, hydrophobically modified hydroxyethylcellulose, and cross-linked acrylate polymers such as Carbomer, hydrophobically modified polyethers) can be included in capsule compositions described above, either in the oil core or capsule wall, or in the capsule slurry outside the capsules. Optionally, silicas, either hydrophobic or hydrophilic, can be included at a concentration from about 0.01% to about 20%, more preferable from 0.5% to about 5%, by the weight of the capsule compositions. Examples of hydrophobic silicas include silanols, surfaces of which are treated with halogen silanes, alkoxysilanes, silazanes, and siloxanes, such as SIPERNAT D17, AEROSIL R972 and R974 available from Degussa. Exemplary hydrophilic silicas are AEROSIL 200, SIPERNAT 22S, SIPERNAT 505 (available from Degussa), and SYLOID 244 (available from Grace Davison).

One or more humectants are optionally included to hold water in the capsule compositions for a long period of time. They constitutes from about 0.01% to about 25% (e.g., 1% to 5%) by weight of the capsule composition. Examples include glycerin, propylene glycol, alkyl phosphate esters, quaternary amines, inorganic salts (e.g., potassium polymetaphosphate, sodium chloride, etc.), polyethylene glycols, and the like.

Further suitable humectants, as well as viscosity control/suspending agents, are disclosed in U.S. Pat. Nos. 4,428,869, 4,464,271, 4,446,032, and 6,930,078. Details of hydrophobic silicas as a functional delivery vehicle of active materials other than a free flow/anticaking agent are disclosed in U.S. Pat. Nos. 5,500,223 and 6,608,017.

In some embodiments, one or more pH modifiers are included in a capsule composition to adjust the pH value of the capsule slurry and/or the oil core. The pH modifiers can also assist in the formation of capsule walls by changing the reaction rate of the crosslinking reactions that form the capsule walls. Exemplary pH modifiers include metal hydroxides (e.g., LiOH, NaOH, KOH, and Mg(OH)₂), metal carbonates and bicarbonates (CsCO₃ Li₂CO₃, K₂CO₃, NaHCO₃, and CaCO₃), metal phosphates/hydrogen phosphates/dihydrogen phosphates, metal sulfates, ammonia, mineral acids (HCl, H₂SO₄, H₃PO₄, and HNO₃), carboxylic acids (e.g., acetic acid, citric acid, lactic acid, benzoic acid, and sulfonic acids), and amino acids.

The capsule compositions of this invention can also include one or more non-confined unencapsulated benefit agents from about 0.01 weight % to about 50 weight %, more preferably from about 5 weight % to about 40 weight %.

Optionally, an emulsifier (i.e., nonionic such as polyoxyethylene sorbitan monostearate (e.g., TWEEN 60), anionic such as sodium oleate, zwitterionic such as lecithins) from about 0.01 weight % to about 25 weight %, more preferably from about 5 weight % to about 10 weight % can be included.

A capsule deposition aid from about 0.01 weight % to about 25 weight %, more preferably from about 5 weight % to about 20 weight % can be included. The capsule deposition aid can be added during the preparation of the capsules or it can be added after the capsules have been made.

These deposition aids are used to aid in deposition of capsules to surfaces such as fabric, hair or skin. These include anionically, cationically, nonionically, or amphoteric water-soluble polymers. Those skilled in the art would appreciate that the charge of these polymers can be adjusted by changing the pH, depending on the product in which this technology is to be used. Any suitable method for coating the deposition aids onto the encapsulated fragrance materials can be used. The nature of suitable polymers for assisted capsule delivery to interfaces depends on the compatibility with the capsule wall chemistry since there has to be some association to the capsule wall. This association can be through physical interactions, such as hydrogen bonding, ionic interactions, hydrophobic interactions, electron transfer interactions or, alternatively, the polymer coating could be chemically (covalently) grafted to the capsule or particle surface. Chemical modification of the capsule or particle surface is another way to optimize anchoring of the polymer coating to capsule or particle surface. Furthermore, the capsule and the polymer need to be compatible with the chemistry (polarity, for instance) of the desired interface. Therefore, depending on which capsule chemistry and interface (e.g., cotton, polyester, hair, skin, wool), the polymer can be selected from one or more polymers with an overall zero (amphoteric: mixture of cationic and anionic functional groups) or net positive charge, based on the following polymer backbones: polysaccharides, polypeptides, polycarbonates, polyesters, polyolefinic (vinyl, acrylic, acrylamide, poly diene), polyester, polyether, polyurethane, polyoxazoline, polyamine, silicone, polyphosphazine, polyaromatic, poly heterocyclic, or polyionene, with molecular weight (MW) ranging from about 1,000 to about 1000,000,000, preferably from about 5,000 to about 10,000,000. As used herein, molecular weight is provided as weight average molecular weight.

Particular examples of cationic polymers that can be used to coat the polyurea or polyurethane capsule include, e.g., polysaccharides such as guar, alginates, starch, xanthan, chitosan, cellulose, dextrans, arabic gum, carrageenan, and hyaluronates. These polysaccharides can be employed with cationic modification and alkoxy-cationic modifications such as cationic hydroxyethyl or cationic hydroxypropyl. For example, cationic reagents of choice are 3-chloro-2-hydroxypropyl trimethylammonium chloride or its epoxy version. Another example is graft-copolymers of polyDADMAC on cellulose. Alternatively, polysaccharides can be employed with aldehyde, carboxyl, succinate, acetate, alkyl, amide, sulfonate, ethoxy, propoxy, butoxy, and combinations of these functionalities; or any hydrophobic modification (compared to the polarity of the polysaccharide backbone). The above modifications can be in any ratio and the degree of functionalization can be up to complete substitution of all functionalizable groups, as long as the theoretical net charge of the polymer is zero (mixture of cationic and anionic functional groups) or preferably positive. Furthermore, up to 5 different types of functional groups may be attached to the polysaccharides. Also, polymer graft chains may be differently modified to the backbone. The counterions can be any halide ion or organic counter ion. See U.S. Pat. Nos. 6,297,203 and 6,200,554.

Another source of cationic polymers contain protonatable amine groups so that the overall net charge is zero (amphoteric: mixture of cationic and anionic functional groups) or positive. The pH during use will determine the overall net charge of the polymer. Examples include silk protein, zein, gelatin, keratin, collagen and any polypeptide, such as polylysine.

Further cationic polymers include polyvinyl polymers with up to 5 different types of monomers can be used. The monomers of such polymer have the generic formula:

—C(R₂)(R₁)—CR₂R₃—

wherein, R₁ is any alkane from C₁-C₂₅ or H, wherein the number of double bonds ranges from 0-5, R₁ is an alkoxylated fatty alcohol with any alkoxy carbon-length of C₁-C₂₅, or R₁ is a liquid crystalline moiety that can provide the polymer with thermotropic liquid crystalline properties;

R₂ is H or CH₃; and

R₃ is —Cl, —NH₂ (i.e., polyvinyl amine or its copolymers with N-vinyl formamide.

Such polyvinyl polymers are sold under the name LUPAMIN 9095 by BASF Corporation. Further suitable cationic polymers containing hydroxylalkylvinylamine units, as disclosed in U.S. Pat. No. 6,057,404.

Another class of materials are polyacrylates with up to 5 different types of monomers. Monomers of polyacrylates have the generic formula:

—CH(R₁)—C(R₂)(CO—R₃—R₄)—

wherein, R₁ is any alkane from C₁-C₂₅ or H with number of double bonds from 0-5, R₁ is an alkoxylated fatty alcohol with a C₁-C₂₅ alkyl chain length, or R₁ is a liquid crystalline moiety that provides the polymer with thermotropic liquid crystalline properties;

R₂ is H or CH₃;

R₃ is a C₁-C₂₅ alkyl alcohol or an alkylene oxide with any number of double bonds, or R₃ may be absent such that the C═O bond is (via the C-atom) directly connected to R₄; and

R₄ is —NH₂, —NHR₁, —NR₁R₂, —NR₁R₂R₆ (where R₆=R₁, R₂, or —CH₂—COOH or its salt), —NH—C(O)—, sulfobetaine, betaine, polyethylene oxide, poly(ethyleneoxide/propylene oxide/butylene oxide) grafts with any end group, H, OH, styrene sulfonate, pyridine, quaternized pyridine, alkyl-substituted pyrrolidone or pyridine, pyridine-N-oxide, imidazolinium halide, imidazolium halide, imidazol, piperidine, —OR₁, —OH, —COOH alkali salt, sulfonate, ethoxy sulphate, pyrrolidone, caprolactam, phenyl-R₄ or naphthalene-R₅, where R₄ and R₅ are R₁, R₂, R₃, sulfonic acid or its alkali salt or organic counter ion. Also, glyoxylated cationic polyacrylamides can be used. Typical polymers of choice are those containing the cationic monomer dimethylaminoethyl methacrylate (DMAEMA) or methacrylamidopropyl trimethyl ammonium chloride (MAPTAC). DMAEMA can be found in GAFQUAT and GAFFIX VC-713 polymers from ISP. MAPTAC can be found in BASF's LUVIQUAT PQ11 PN and ISP's GAFQUAT HS100.

Another group of polymers that can be used are those that contain cationic groups in the main chain or backbone. Included in this group are:

i) polyalkylene imines such as polyethylene imine, commercially available as LUPASOL from BASF. Any molecular weight and any degree of crosslinking of this polymer can be used in the present invention;

ii) ionenes as disclosed in U.S. Pat. No. 4,395,541 and U.S. Pat. No. 4,597,962;

iii) adipic acid/dimethyl amino hydroxypropyl diethylene triamine copolymers, such as CARTARETIN F-4 and F-23, commercially available from Sandoz;

iv) polymers of the general formula: —[N(CH₃)₂—(CH₂)ₓ—NH—(CO)—NH—(CH₂)_{y}—N(CH₃)₂)—(CH₂)_{z}—O—(—CH₂)ₚ]ₙ—, with x, y, z, p=1-12, and n according to the molecular weight requirements. Examples are Polyquaternium-2 (MIRAPOL A-15), Polyquaternium-17 (MIRAPOL AD-1), and Polyquaternium-18 (MIRAPOL AZ-1). Other polymers include cationic polysiloxanes and cationic polysiloxanes with carbon-based grafts with a net theoretical positive charge or equal to zero (mixture of cationic and anionic functional groups). This includes cationic end-group functionalized silicones (i.e., Polyquaternium-80). Silicones with general structure: —Si(R₁)(R₂)—O—]ₓ—[Si(R₃)(R₂)—O—]_{y}— where R₁ is any alkane from C₁-C₂₅ or H with number of double bonds from 0-5, aromatic moieties, polysiloxane grafts, or mixtures thereof. R₁ can also be a liquid crystalline moiety that can provide the polymer with thermotropic liquid crystalline properties. R₂ can be H or CH₃; and R₃ can be —R₁-R₄, where R₄ can be —NH₂, —NHR₁, —NR₁R₂, —NR₁R₂R₆ (where R₆=R₁, R₂, or —CH₂—COOH or its salt), —NH—C(O)—, —COOH, —COO— alkali salt, any C₁-C₂₅ alcohol, —C(O)—NH₂ (amide), —C(O)—N(R₂)(R₂′)(R₂″), sulfobetaine, betaine, polyethylene oxide, poly(ethyleneoxide/propylene oxide/butylene oxide) grafts with any end group, H, —OH, styrene sulfonate, pyridine, quaternized pyridine, alkyl-substituted pyrrolidone or pyridine, pyridine-N-oxide, imidazolinium halide, imidazolium halide, imidazol, piperidine, pyrrolidone, caprolactam, sulfonate, ethoxysulphate phenyl-R₅ or naphthalene-R₆ where R₅ and R₆ are R₁, R₂, R₃, sulfonic acid or its alkali salt or organic counter ion. R₃ can also be —(CH₂)ₓ—O—CH₂—CH(OH)—CH₂—N(CH₃)₂—CH₂—COOH and its salts. Any mixture of these R₃ groups can be selected. X and y can be varied as long as the theoretical net charge of the polymer is zero (amphoteric) or positive. In addition, polysiloxanes containing up to 5 different types of monomeric units may be used. Examples of suitable polysiloxanes are found in U.S. Pat. Nos. 4,395,541 4,597,962 and 6,200,554. Another group of polymers that can be used to improve capsule/particle deposition are phospholipids that are modified with cationic polysiloxanes. Examples of these polymers are found in U.S. Pat. No. 5,849,313, WO Patent Application 95/18096A1 and European Patent No. 0737183B1.

Furthermore, copolymers of silicones and polysaccharides and proteins can be used (e.g., those commercially available as CRODASONE brand products).

Another class of polymers includes polyethylene oxide-co-propyleneoxide-co-butylene oxide polymers of any ethylene oxide/propylene oxide/butylene oxide ratio with cationic groups resulting in a net theoretical positive charge or equal to zero (amphoteric). Examples of such polymers are the commercially available TETRONIC brand polymers.

Suitable polyheterocyclic (the different molecules appearing in the backbone) polymers include the piperazine-alkylene main chain copolymers disclosed by Kashiki and Suzuki (1986) Ind. Eng. Chem. Fundam. 25:120-125.

Table 2 below shows polyquaternium polymers that can be used as deposition aids in this invention.

TABLE 2
Deposition Aids-Cationic Polyquaternium Polymers

| Polyquatemium | Description | Commercial Products |
|---|---|---|
| 1 | Ethanol, 2,2′,2″-nitrilotris-, polymer with | Polyquad (Alcon) |
| | 1,4-dichloro-2-butene and N,N,N′,N′- | |
| | tetramethyl-2-butene-1,4-diamine | |
| 2 | Poly[bis(2-chloroethyl) ether-alt-1,3-bis[3- | Mirapol A-15 |
| | (dimethylamino)propyl]urea] | |
| 4 | Hydroxyethyl cellulose dimethyl | Celquat L-200, H-100, |
| | diallylammonium chloride copolymer; | L-200 |
| | Diallyldimethylammonium chloride- | |
| | hydroxyethyl cellulose copolymer | |
| 5 | Copolymer of acrylamide and quaternized | Merquat 5, RETEN |
| | dimethylammoniumethyl methacrylate | (Hercules) |
| 6 | Poly(diallyldimethylammonium chloride) | Merquat 100, 106, |
| | | Mirapol 100 |
| 7 | Copolymer of acrylamide and | Merquat 550, 550L, |
| | diallyldimethylammonium chloride | 550PR, S, 7SPR, 740, |
| | | 2200, Mirapol 550, |
| | | Polyquart 770/NA, |
| | | Conditioneze 7 |
| 8 | Methyl and Stearyl Dimethylaminoethyl | |
| | Methacrylate Quaternized with Dimethyl | |
| | Sulfate | |
| 9 | Polydimethylaminoethyl Methacrylate | |
| | Quaternized with Methyl Bromide | |
| 10 | Quaternized hydroxyethyl cellulose | Merquat 10, Celquat |
| | | SC-230M, SC-240C, |
| | | SC-140C, Ucare |
| | | Polymer |
| 11 | Copolymer of vinylpyrrolidone and | Luviquat PQ 11PN, |
| | quaternized dimethylaminoethyl | Gafquat 775N, 440, |
| | methacrylate | 734, 775 |
| 12 | 2-Propenoic Acid, 2-Methyl-, Decahydro- | |
| | 1,4-Dimethyl-7-(1-Methylethyl)-1- | |
| | Phenanthrenyl)Methyl Ester, Polymer with | |
| | 2-(Diethylamino)Ethyl 2-Methyl-2- | |
| | Propenoate and Ethyl 2-Methyl-2- | |
| | Propenoate, compd. with Dimethyl Sulfate | |
| 13 | 2-Propenoic Acid, 2-Methyl-, 2-(Diethyl- | |
| | amino)Ethyl Ester, Polymer with Ethyl 2- | |
| | Methyl-2-Propenoate and 9-Octadecenyl | |
| | 2-Methyl-2-Propenoate, compd. with | |
| | Dimethyl Sulfate | |
| 14 | Ethanaminium, N,N,N-Trimethyl-2-[(2- | |
| | Methyl-1-Oxo-2-Propenyl)Oxy]-, Methyl | |
| | Sulfate, Homopolymer | |
| 15 | Ethanaminium, N,N,N-Trimethyl-2-[(2- | Rohagit KF 720F |
| | Methyl-1-Oxo-2-Propenyl)Oxy]-,Chloride, | (Rohm GmbH) |
| | Polymer with 2-Propenamide | |
| 16 | Copolymer of vinylpyrrolidone and | Luviquat FC 370, HM |
| | quaternized vinylimidazole | 552, Style, FC 550, |
| | | Excellence |
| 17 | Poly(Oxy-1,2-Ethanediyl (Dimethyl- | Mirapol AD |
| | iminio)-1,3-Propanediylimino(1,6-Dioxo- | |
| | 1,6-Hexanediyl)Imino-1,3-Propanediyl- | |
| | (Dimethyliminio)-1,2-Ethanediyl | |
| | Dichloride | |
| 18 | Poly[oxy-1,2-ethanediyl(dimethyliminio)- | Luviquat 500 |
| | 1,3-propanediylimino-(1,6-dioxo-1,6- | |
| | heptanediyl)imino-1,3-propanediyl- | |
| | (dimethyliminio)-1,2-ethanediyl | |
| | dichloride] | |
| 19 | Ethenol, polymer with | Arlatone PQ-220 (ICI |
| | aminomethyloxirane | Americas) |
| 20 | Ethenyl octadecyl ether, polymer with | Arlatone PQ-225 |
| | aminomethyloxirane | |
| 22 | Copolymer of Acrylic Acid and | Merquat 280, 281, |
| | Diallyldimethylammonium Chloride | 280SD, 295 |
| 24 | Cellulose, 2-[2-Hydroxy-3-(Trimethyl- | Quatrisoft Polymer LM- |
| | ammonio)Propoxy]Ethyl Ether, Chloride | 200 (Dow Chemical) |
| | (Similar to PQ-10) | |
| 27 | Hexanediamide, N,N-bis(3-(Dimethyl- | |
| | amino)Propyl)-, Polymer with N,N′-bis(3- | |
| | Dimethylamino)Propyl Urea and 1,1′- | |
| | Oxybis(2-Chloroethane), Block | |
| 28 | Copolymer of vinylpyrrolidone and meth- | Gafquat HS-100, |
| | acrylamidopropyl trimethylammonium | Conditioneze NT-10 |
| 29 | Chitosan, 2,3-Dihydroxypropyl-2- | Quaternized Chitosan |
| | Hydroxy-3-(Trimethylammonio)Propyl | |
| | Ether, Chloride | |
| 30 | Ethanaminium, NCarboxymethyl)-N,N- | Mexomere PX |
| | Dimethyl-2-((2-Methyl-1-Oxo-2- | (Chimex) |
| | Propenyl)Oxy)-, Inner Salt, | |
| | Polymer with Methyl 2-Methyl- | |
| | 2-Propenoate | |
| 31 | 2-Propenenitrile, Homopolymer, | Hypan QT100 (Lipo) |
| | Hydrolyzed, Block, Reaction | |
| | Products with N,N-Dimethyl-1, | |
| | 3-Propanediamine, Di-Et | |
| | Sulfate-Quaternized | |
| 32 | Poly(acrylamide 2-methacryloxyethyl- | Cosmedia CTC (Cognis |
| | trimethyl ammonium chloride) | GmbH)-PQ-32 + |
| | | other, Salcare SC92 |
| | | (Ciba Corp.) PQ-32 + |
| | | other |
| 33 | Ethanaminium, N,N,N-Trimethyl- | Lanoquat DES-50, |
| | 2-[1-Oxo-2-Propenyl)Oxy]-, | Ultimer CG-200 |
| | Chloride, Polymer with 2- | (Nalco), Sepigel Quat33 |
| | Propenamide | (Seppic)-PQ-33 + |
| | | other |
| 34 | Poly(diethyliminio-1,3-propanediyldi- | Mexomere PAK |
| | methyliminio-1,3-propanediyl dibromide) | (Chimex) |
| 35 | Ethanaminium, N-carboxymethyl-N,N- | Plex 3074 L (Rohm |
| | dimethyl-2-(2-methyl-1-oxo-2- | GmbH) |
| | propenyloxy)-, inner salt, polymer with | |
| | N,N,N-trimethyl-2-(2-methyl-1-oxo-2- | |
| | propenyloxy)ethanaminium methyl sulfate | |
| 36 | 2-Propenoic Acid, 2-Methyl-, | Plex 4739L (Rohm |
| | 2-(Dimethylamino)Ethyl Ester, | GmbH) |
| | Polymer with Methyl 2-Methyl- | |
| | 2-Propenoate, compd. with | |
| | Dimethyl Sulfate | |
| 37 | N,N,N-Trimethyl-2-[(Methyl-1- | Ultragel 300 (Cognis), |
| | Oxo-2-Propenyl)Oxy]Ethanaminium | Synthalen CN, CR, CU |
| | Chloride, Homopolymer | (3V Group), Syntran PC |
| | | 5320 (Interpolymer) |
| 39 | 2-Propen-1-aminium, N,NDimethyl-N-2- | Merquat 3940, PLUS- |
| | Propenyl-, Chloride, Polymer with 2- | 3330, PLUS-3331, |
| | Propenamide and 2-Propenoic Acid | 3331PR |
| 42 | Poly[oxyethylene(dimethyliminio)ethylene | Busan 1507 (Buckman |
| | (dimethylimino)ethylene dichloride] | Labs) |
| 43 | polymeric quaternary ammonium salt | Genamin PQ 43 |
| | formed from acrylamide, | (Clariant |
| | acrylamidopropyltrimonium chloride, 2- | Functional Chemicals), |
| | amidopropylacrylamide sulfonate, and | Bozequat 4000 |
| | DMAPA monomers | (Clariant) |
| 44 | Poly(2-oxopyrrolidin-1-ylethylene, 3- | Luviquat Ultracare, MS |
| | methylimidazolium-1-ylethylene methyl | 370 (BASF), Softenol |
| | sulfate) | PQ44 (Zdchimmer & |
| | | Schwarz Italianat SpA) |
| 45 | Glycine, N-methyl-N-[2-[(2-methyl-1-oxo- | Plex 3073L (Rohm |
| | 2-propenyl)oxy]ethyl]-, polymer with 2- | GmbH) |
| | (dimethylamino)ethyl 2-methyl-2- | |
| | propenoate, compound with dimethyl | |
| | sulfate | |
| 46 | 1H-Imidazolium, 1-Ethenyl-3-Methyl-, | Luviquat Hold |
| | Methyl Sulfate, Polymer with 1-Ethenyl- | |
| | hexahydro-2H-Azepin-2-one and 1- | |
| | Ethenyl-2-Pyrrolildinone | |
| 47 | 1-Propanaminium, N,N,NTrimethyl-3-((2- | Merquat 2001, 2001N |
| | Methyl-1-Oxo-2-Propenyl)Amino)-, | |
| | Chloride, Polymer with Methyl 2- | |
| | Propenoate and 2-Propenoic Acid | |
| 48 | olymeric quaternary ammonium salt of | Plascize L-450 (Goo |
| | formed from methacryloyl ethyl betaine, 2- | Chemical) |
| | hydroxyethyl methacrylate and | |
| | methacryloyl ethyl trimethyl ammonium | |
| | chloride | |
| 49 | polymeric quaternary ammonium salt | Plascize L-440 |
| | formed by the reaction of methacryloyl | (Goo Chemical) |
| | ethyl betaine, PEG-9 methacrylate and | |
| | methacryloyl ethyl trimethyl ammonium | |
| | chloride | |
| 50 | Carboxylatoethyldimethylammonioethyl 2- | Plascize L-401 (Goo |
| | methyl-2-propenoate homopolymer | Chemical) |
| 51 | 3,5,8-Triox-4-Phosphaundec-10- | Lipidure PMB |
| | en-1-aminium, 4-Hydroxy-N,N, | (NOF) |
| | N,10-Tetramethyl-9-Oxo, Inner | |
| | Salt, 4-Oxide, Polymer with | |
| | Butyl 2-Methyl-2-Propenoate | |
| 53 | Acrylic Acid/Acrylamide/Methacryl- | Merquat 2003PR |
| | amidopropyltrimonium Chloride | |
| | Copolymer | |
| 54 | Aspartic acid, polymer with C6-18 | Quilty-Hy (Mitsui) |
| | alkylamine, 3-dimethylaminopropylamine | |
| | and sodium chloroacetate | |
| 55 | 1-Dodecanaminium, N,NDimethyl-N-[3- | Styreze W |
| | [(2-Methyl-1-Oxo-2-Propenyl)-Amino- | |
| | Propyl]-, Chloride, Polymer with N-[3- | |
| | (Dimethylamino)Propyl]-2-Methyl-2- | |
| | Propenamide and 1-Ethenyl-2- | |
| | Pyrrolidinone | |
| 56 | 5-Isocyanato-1-(iso cyanatomethyl)-1,3,3- | Hairrol UC-4 (Sanyo |
| | trimethylcyclohexane, polymer with 1,3- | Chemical) |
| | butanediol and bis(2-hydroxyethyl)di- | |
| | methylammonium methyl sulfate | |
| 57 | 12-Hydroxy-9(Z)-octadecenamidopropyl- | Zenigloss Q (Zenitech) |
| | trimethylammonium chloride, polymers | |
| | with ricinus communis (castor) oil, | |
| | isooctdecanoic acid and butandioic acid | |
| 58 | 2-Propenoic Acid, Methyl Ester, Polymer | Lowenol Conditioner |
| | with 2,2-Bis[(2-Propenyloxy)Methyl]-1- | PWW |
| | Butanol and Diethenylbenzene, Reaction | (Lowenstein)-PQ-58 |
| | Products with N,NDimethyl-1,3-Propane- | and Wheat Protein |
| | diamine, Chloromethane-Quaternized | |
| 59 | Poly(20,25-dioxo-2,5,10,15,18-penta- | Crodasorb UV-HPP |
| | methyl-10-(2-hydroxy-3-(3-(3-phenyl-2- | (Croda, Inc.)-PQ-59 |
| | propenamido)propyldimethylammonio) | and Butylene Glycol |
| | propyl)-10-azonia-1,4,7,13,16,19-hexaoxa- | |
| | pentacosanediyl) chloride | |
| 60 | 9-Octadecenoic Acid, 12-Hydroxy-, [(2- | Polylipid PPI-RC |
| | Hydroxyethyl)-Imino]Di-2,1-Ethanediyl | (Alzo/Bernel)-PQ-60 |
| | Ester, Polymer with 5-Isocyanato-1- | and Propylene Glycol |
| | (Isocyanatomethyl)-1,3,3-Trimethyl- | |
| | cyclohexane, Compd. with Diethyl Sulfate | |
| 61 | 2-Methyl-2-propenoyloxyethyl N,N,N- | Lipidure-S (NOF) |
| | trimethylammonioethyl phosphate inner | |
| | salt, polymer with octadecyl 2-methyl-2- | |
| | propenoate | |
| 62 | Polymeric quaternary ammonium salt of | Nanoaquasome (Amore |
| | butyl methacrylate, polyethylene glycol | Pacific/Kyung-do) |
| | methyl ether methacrylate, ethylene glycol | |
| | dimethacrylate and 2-methacryloylethyl | |
| | trimonium chloride with 2,2′-azobis(2- | |
| | methyl propionamidine)dihydrochloride | |
| 63 | polymeric quaternary ammonium salt | Finquat (Innospec), |
| | formed by acrylamide, acrylic acid and | Octacare PQ63 |
| | ethyltrimonium chloride acrylate | (Innospec Edison, NJ), |
| | | OF-308 (WSP Chemical |
| | | & Technology) |
| 64 | 2-Methyl-2-propenoyloxyethyl N,N,N- | Lipidure-C (NOF) |
| | trimethylammonioethyl phosphate inner | |
| | salt, polymer with 2-hydroxy-3-(2-methyl- | |
| | 2-propenoyl)oxypropyltrimethyl- | |
| | ammonium chloride | |
| 65 | 2-Methyl-2-propenoyloxyethyl N,N,N- | Lipidure-A (NOF) |
| | trimethylammonioethyl phosphate inner | |
| | salt, polymer with butyl 2-methyl-2- | |
| | propenoate and sodium 2-methyl-2- | |
| | propenoate | |
| 66 | 5-Isocyanato-1-(isocyanatomethyl)-1,3,3- | WBR-2925C (Taisei)- |
| | trimethylcyclohexane, polymer with | PQ-66 and Methyl |
| | di(hydroxypolymethylene) benzene- | Pyrrolidone |
| | dicarboxylate and ethylbis(2-hydroxy- | |
| | ethyl)methylammonium ethyl sulfate | |
| 67 | 2-Hydroxyethyl cellulose ether, reaction | Softcat (Dow Chemical) |
| | products with N,N,N-trimethyl-N- | |
| | oxiranylmethylammonium chloride and N- | |
| | dodecyl-N,N-dimethyl-N- | |
| | oxiranylmethylammonium chloride | |
| 68 | 1-Ethenyl-2-pyrrolidinone, polymer with | Luviquat Supreme |
| | 1-ethenylimidazole and 1-ethenyl-3- | |
| | methylimidazolium methyl sulfate | |
| 69 | polymeric quaternary ammonium salt | Aquastyle 100, 300 |
| | composed of vinyl caprolactam, | (ISP) |
| | vinylpyrrolidone, dimethylaminopropyl | |
| | methacrylamide (DMAPA), and | |
| | methacryloylaminopropyl lauryldimonium | |
| | chloride | |
| 70 | polymeric quaternary ammonium salt | Lustreplex (Croda) |
| | consisting of an ethoxylated, propoxylated | |
| | stearyl amine condensed with adipic acid | |
| | and dilinoleic acid and quaternized with | |
| | dimethyl sulfate | |
| 71 | | ColaMoist 300P |
| | | (Colonial Chemical Inc) |
| 72 | polymeric quaternary ammonium salt of | Mirustyle CP (Croda) |
| | hydroxethylcellulose reacted with a coco- | |
| | alkyl dimethyl ammonium substituted | |
| | epoxide | |
| 73 | polymeric quaternary ammonium salt | Diaformer C-802, C- |
| | consisting of propyltrimonium chloride | 823 (Mitsubishi Chem), |
| | acrylamide, ethyltrimonium chloride | Diasleek C-802, C-823 |
| | methacrylate and dimethylacrylamide | (Mitsubishi Chem) |
| | monomers; Propanaminium, N,N,N- | |
| | trimethyl-3-(2-propenamido)-, chloride, | |
| | polymer with N,N,N-trimethyl-2-(2- | |
| | methyl-2-propenoyloxy)ethanaminium | |
| | chloride and N,N-dimethyl-2-propenamide | |
| 74 | | Mirapol PB 20 (Rhodia) |
| | | Polycare Boost |
| | | (Rhodia) |
| 75 | O-(2-Hydroxy-2-trimethylammonio- | Amylomer Cat |
| | propyl)starch chloride, reaction products | 220EMU (Grafe |
| | with O-(3-dodecyldimethylammonio-2- | Chemie) |
| | hydroxypropyl)starch chloride | |
| 76 | | Mirapol AT-1 (Rhodia) |
| 77 | Cocoglucoside Crosspolymer | Colonial Poly SugaQuat |
| | Hydroxypropyltrimonium | TM-8610P (Colonial |
| | Chloride | Chemical Inc) |
| 78 | Decylglucoside Crosspolymer | Colonial Poly SugaQuat |
| | Hydroxypropyl Laurdimonium | L-1010P (Colonial |
| | Chloride | Chemical Inc) |
| 79 | Decylglucoside Crosspolymer | Colonial Poly SugaQuat |
| | Hydroxypropyl Steardimonium | S-1010P (Colonial |
| | Chloride | Chemical Inc) |
| 80 | Laurylglucoside Crosspolymer | Colonial Poly SugaQuat |
| | Hydroxypropyl Laurdimonium | L-1210P (Colonial |
| | Chloride | Chemical Inc) |
| 81 | Laurylglucoside Crosspolymer | Colonial Poly SugaQuat |
| | Hydroxypropyl Steardimonium | S-1210P (Colonial |
| | Chloride | Chemica Inc) |
| 82 | Laurylglucoside Crosspolymer | Colonial Poly SugaQuat |
| | Hydroxypropyltrimonium | TM-1218P (Colonial |
| | Chloride | Chemical Inc) |
| 84 | polymeric quaternary ammonium salt of | Diasleek C-824 |
| | acrylamidopropyltrimethylammonium | (Mitsubishi Chemical) |
| | chloride, trimethylaminoethyl | |
| | methacrylate, dimethylacrylamide and | |
| | hydroxyethylmethacrylate | |
| 85 | polymeric quaternary ammonium salt of | Diasleek C-825 |
| | acrylamidopropyltrimethylammonium | (Mitsubishi Chemical) |
| | chloride, dimethylacrylamide and | |
| | hydroxyethylmethacrylate | |
| 86 | polymeric quaternary ammonium salt of | Luvigel Advanced |
| | vinylpyrrolidone, 1-methyl-3- | (BASF) |
| | vinylimidazoline chloride, vinylimidazole | |
| | and methacrylic acid | |
| 87 | polymeric quaternary ammonium salt of | Luviquat Sensation |
| | vinylpyrrolidone, vinylimidazole and | (BASF) |
| | diallyldimethyl ammonium chloride | |
| 88 | Poly(Dilinoleyldimonium | ColaQuat PDQ |
| | hydroxypropyl)chlorides) | (Colonial Chemical Inc) |
| 89 | polymeric quaternary ammonium salt | (BASF) |
| | prepared by the reaction of t-butyl acrylate, | |
| | vinyl pyrolidone, dimethylaminopropyl | |
| | methacrylamide, methacrylic acid and | |
| | ethyldimethyl[2-[(2-methyl-1- | |
| | oxoallyl)oxy]ammonium ethyl sulfate, | |
| | neutralized with orthophosphoric acid | |
| 90 | polymeric quaternary ammonium salt of | Hymoquat AK325R |
| | acrylamide and hydroxyethylcellulose | (Hymo Corporation) |
| | quaternized with diallyldimethyl | |
| | ammonium chloride | |
| 91 | polymeric quaternary ammonium salt of | Syntran 5500 |
| | hydroxypropyl methacrylate and | (Interpolymer)-PQ-91 |
| | polyethylene glycol methacrylate | and PA |
| | quaternized with ethyltrimonium chloride | |
| | methacrylate | |
| 92 | GLYCERYLAMIDOETHYL | Ceracute-G (NOF) |
| | METHACRYLATE/STEARYL | |
| | METHACRYLATE COPOLYMER | |
| 94 | polymeric quaternary ammonium salt | (Toho) |
| | consisting of acrylamide, dimethyl diallyl | |
| | ammonium chloride and | |
| | methacrylamidopropyltrimonium chloride | |
| | monomers | |
| 95 | copolymer of Zea Mays (Corn) Starch, | Polyquart Ecoclean |
| | Acrylic Acid and | (Cognis) |
| | acrylamidopropyltrimethylammonium | |
| | chloride monomers | |
| 98 | | (Cognis GmbH) |
| 101 | | Deposilk Q1 (Air |
| | | Products) |

Other suitable deposition aids include those described in US 2013/0330292, US 2013/0337023, US 2014/0017278.

Additional polymeric core modifiers are also contemplated. It has been found that the addition of hydrophobic polymers to the core can also improve stability by slowing diffusion of the active material from the core. The level of polymer is normally less than 80% of the core by weight, preferably less than 50%, and more preferably less than 20%. The basic requirement for the polymer is that it be miscible or compatible with the other components of the core, namely the active material and other solvent. Preferably, the polymer also thickens or gels the core, thus further reducing diffusion. These additional polymeric core modifiers include copolymers of ethylene; copolymers of ethylene and vinyl acetate (ELVAX polymers by DOW Corporation); copolymers of ethylene and vinyl alcohol (EVAL polymers by Kuraray); ethylene/acrylic elastomers such as VALNAC polymers by Dupont; polyvinyl polymers, such as polyvinyl acetate; alkyl-substituted cellulose, such as ethyl cellulose (ETHOCEL made by DOW Corporation) and hydroxypropyl celluloses (KLUCEL polymers by Hercules); cellulose acetate butyrate available from Eastman Chemical; polyacrylates (e.g., AMPHOMER, DEMACRYL LT and DERMACRYL 79, made by National Starch and Chemical Company, the AMERHOLD polymers by Amerchol Corporation, and ACUDYNE 258 by ISP Corporation); copolymers of acrylic or methacrylic acid and fatty esters of acrylic or methacrylic acid such as INTELIMER POLYMERS made by Landec Corporation (see also U.S. Pat. Nos. 4,830,855, 5,665,822, 5,783,302, 6,255,367 and 6,492,462); polypropylene oxide; polybutylene oxide of poly(tetrahydrofuran); polyethylene terephthalate; polyurethanes (DYNAM X by National Starch); alkyl esters of poly(methyl vinyl ether); maleic anhydride copolymers, such as the GANTREZ copolymers and OMNIREZ 2000 by ISP Corporation; carboxylic acid esters of polyamines, e.g., ester-terminated polyamides (ETPA) made by Arizona Chemical Company; polyvinyl pyrrolidone (LUVISKOL series of BASF); block copolymers of ethylene oxide, propylene oxide and/or butylenes oxide including, e.g., PLURONIC and SYNPERONIC polymers/dispersants by BASF. Another class of polymers include polyethylene oxide-co-propyleneoxide-co-butylene oxide polymers of any ethylene oxide/propylene oxide/butylene oxide ratio with cationic groups resulting in a net theoretical positive charge or equal to zero (amphoteric). The general structure is:

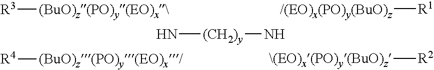

where R¹, R², R³, and R⁴ are independently H or any alkyl or fatty alkyl chain group. Examples of such polymers are the commercially known as TETRONICS by BASF Corporation.

Sacrificial core ingredients can also be included. These ingredients are designed to be lost during or after manufacture and include, but are not limited to, highly water soluble or volatile materials.

In addition to the capsules and adjunct materials described above, the delivery system of this invention can contain one or more other delivery compositions such as polymer-assisted delivery compositions (see U.S. Pat. No. 8,187,580), fiber-assisted delivery compositions (US 2010/0305021), cyclodextrin host guest complexes (U.S. Pat. No. 6,287,603 and US 2002/0019369), pro-fragrances (WO 2000/072816 and EP 0 922 084), membrane delivery systems (U.S. Pat. No. 4,948,047), and any combination thereof.

As used herein olfactory effective amount is understood to mean the amount of compound in the delivery system the individual components will contribute to its particular olfactory characteristics, but the olfactory effect of the delivery system will be the sum of the effects of each of the individual components. Thus, the capsule delivery systems of this invention can be used to alter the aroma characteristics of a consumer product, e.g., a fine perfume, by modifying the olfactory reaction contributed by another ingredient in the consumer product. The amount will vary depending on many factors including other ingredients, their relative amounts and the effect that is desired.

In accordance with some embodiments of this invention, the capsules can be cured at a temperature in the range of, e.g., 55° C. to 130° C. (e.g., 55° C. to 90° C., 55° C. to 75° C., and 90° C. to 130° C.) for 1 minute to 10 hours (e.g., 0.1 hours to 5 hours, 0.2 hours to 4 hours and 0.5 hours to 3 hours). A skilled person in the art can determine, without undue experiments, the curing temperature, duration, and the heating rate.

Not to be bound by any theory, it is believed that there is a direct relationship between higher cure temperature and less leaching of active material from the capsule. In accordance with one embodiment, the capsules are cured at a temperature at or above 100° C. The retention capabilities of the capsules are thus improved. In another embodiment, the capsules are cured at or above 110° C. In still another embodiment, the capsules are cured at or above 120° C.

To obtain capsules with more leaching of the active material, certain embodiments of this invention provide for a cure temperature of less than 100° C. In some embodiments, the cure temperature is at or less than 90° C. In other embodiments, the cure temperature is at or less than 80° C.

In one embodiment, the capsules are heated to a target cure temperature at a linear rate of 0.5 to 2° C. per minute (e.g., 1 to 5° C. per minute, 2 to 8° C. per minute, and 2 to 10° C. per minute) over a period of 1 to 60 minutes (e.g., 1 to 30 minutes). The following heating methods may be used: conduction for example via oil, steam radiation via infrared, and microwave, convection via heated air, steam injection and other methods known by those skilled in the art. The target cure temperature used herein refers to the minimum temperature in degrees Celsius at which the capsules may be cured to retard leaching.

Applications.

The delivery systems of the present invention are well-suited for use, without limitation, in the following products:
a) Household products
   i. Liquid or Powder Laundry Detergents which can use the present invention include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818
   ii. Unit Dose Pouches, Tablets and Capsules such as those described in EP 1 431 382 A1, US 2013/0219996 A1, US 2013/0284637 A1, and U.S. Pat. No. 6,492,315. These unit dose formulations can contain high concentrations of a functional material (e.g., 5-100% fabric softening agent or detergent active), fragrance (e.g., 0.5-100%, 0.5-40%, and 0.5-15%), and flavor (e.g., 0.1-100%, 0.1-40%, and 1-20%). They can contain no water to limit the water content as low as less than 30% (e.g., less than 20%, less than 10%, and less than 5%).
   iii. Scent Boosters such as those described in U.S. Pat. No. 7,867,968, U.S. Pat. No. 7,871,976, U.S. Pat. No. 8,333,289, US 2007/0269651 A1, and US2014/0107010 A1.
   iv. Fabric Care Products such as Rinse Conditioners (containing 1 to 30 weight % of a fabric conditioning active), Fabric Liquid Conditioners (containing 1 to 30 weight % of a fabric conditioning active), Tumble Drier Sheets, Fabric Refreshers, Fabric Refresher Sprays, Ironing Liquids, and Fabric Softener Systems such as those described in U.S. Pat. Nos. 6,335,315, 5,674,832, 5,759,990, 5,877,145, 5,574,179, 5,562,849, 5,545,350, 5,545,340, 5,411,671, 5,403,499, 5,288,417, 4,767,547 and 4,424,134
   v. Liquid dish detergents such as those described in U.S. Pat. Nos. 6,069,122 and 5,990,065
   vi. Automatic Dish Detergents such as those described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562
   vii. All-purpose Cleaners
   viii. Bathroom Cleaners
   ix. Bath Tissue
   x. Rug Deodorizers
   xi. Candles
   xii. Room Deodorizers
   xiii. Floor Cleaners
   xiv. Disinfectants
   xv. Window Cleaners
b) Household Devices
   i. Paper towels
   ii. Disposable Wipes
c) Baby Care Products
   i. Diaper Rash Cream/Balm
   ii. Baby Powder
d) Baby Care Devices
   i. Diapers
   ii. Bibs
   iii. Wipes
e) Oral Care Products. Tooth care products (as an example of preparations according to the invention used for oral care) generally include an abrasive system (abrasive or polishing agent), for example silicic acids, calcium carbonates, calcium phosphates, aluminum oxides and/or hydroxylapatites, surface-active substances, for example sodium lauryl sulfate, sodium lauryl sarcosinate and/or cocamidopropylbetaine, humectants, for example glycerol and/or sorbitol, thickening agents, for example carboxymethyl cellulose, polyethylene glycols, carrageenan and/or Laponite®, sweeteners, for example saccharin, taste correctors for unpleasant taste sensations, taste correctors for further, normally not unpleasant taste sensations, taste-modulating substances (for example inositol phosphate, nucleotides such as guanosine monophosphate, adenosine monophosphate or other substances such as sodium glutamate or 2-phenoxypropionic acid), cooling active ingredients, for example menthol derivatives, (for example L-menthyllactate, L-menthylalkylcarbonates, menthone ketals, menthane carboxylic acid amides), 2,2,2-trialkylacetic acid amides (for example 2,2-diisopropylpropionic acid methyl amide), icilin and icilin derivatives, stabilizers and active ingredients, for example sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetylpyridinium chloride, aluminum lactate, potassium citrate, potassium nitrate, potassium chloride, strontium chloride, hydrogen peroxide, flavorings and/or sodium bicarbonate or taste correctors.
   i. Tooth Paste. An exemplary formulation as follows:
      1. calcium phosphate 40-55%
      2. carboxymethyl cellulose 0.8-1.2%
      3. sodium lauryl sulfate 1.5-2.5%
      4. glycerol 20-30%
      5. saccharin 0.1-0.3%
      6. flavor oil 1.0-2.5%
      7. water q.s. to 100%
         A typical procedure for preparing the formulation includes the steps of (i) mixing by a blender according to the foregoing formulation to provide a toothpaste, and (ii) adding a composition of this invention and blending the resultant mixture till homogeneous.
   ii. Tooth Powder
   iii. Oral Rinse
   iv. Tooth Whiteners
   v. Denture Adhesive
f) Hand Sanitizer
g) Antiinflammatory balms, ointment or spray
h) Health Care Devices
   i. Dental Floss
   ii. Toothbrushes
i) Feminine Hygiene Products such as Tampons, Feminine Napkins and Wipes, and Pantiliners
j) Personal Care Products: Cosmetic or pharmaceutical preparations, e.g., a “water-in-oil” (W/O) type emulsion, an “oil-in-water” (O/W) type emulsion or as multiple emulsions, for example of the water-in-oil-in-water (W/O/W) type, as a PIT emulsion, a Pickering emulsion, a micro-emulsion or nano-emulsion; and emulsions which are particularly preferred are of the “oil-in-water” (O/W) type or water-in-oil-in-water (W/O/W) type. More specifically,
   i. Personal Cleansers (Bar Soap, Body Wash, Shower Gel)
   ii. Sunscreen (Spray or lotion)
   iii. Insect repellants
   iv. Antibacterial ointments or creams
   v. Sensates
   vi. Deodorants and Antiperspirants including aerosol antiperspirant, stick antiperspirant, roll-on antiperspirant, emulsion spray antiperspirant, clear emulsion stick antiperspirant, soft solid antiperspirant, emulsion roll-on antiperspirant, clear emulsion stick antiperspirant, opaque emulsion stick antiperspirant, clear gel antiperspirant, clear stick deodorant and spray deodorant.
   vii. Wax-based Deodorant. An exemplary formulation as follows:
      1. Parafin Wax 10-20%
      2. Hydrocarbon Wax 5-10%
      3. White Petrolatum 10-15%
      4. Acetylated Lanolin Alcohol 2-4%
      5. Diisopropyl Adipate 4-8%
      6. Mineral Oil 40-60%
      7. Preservative (as needed)
         The formulation is prepared by (i) mixing the above ingredients, (ii) heating the resultant composition to 75° C. until melted, (iii) with stirring, adding 4% cryogenically ground polymer containing a fragrance while maintaining the temperature 75° C., and (iv) stirring the resulting mixture in order to ensure a uniform suspension while a composition of this invention is added to the formulation.
   viii. Glycol/Soap Type Deodorant. An exemplary formulation as follows:
      1. Propylene Glycol 60-70%
      2. Sodium Stearate 5-10%
      3. Distilled Water 20-30%
      4. 2,4,4-Trichloro-2′-Hydroxy Diphenyl Ether, manufactured by the Ciba-Geigy Chemical Company and a Trademark of the Ciba-Geigy Chemical Company) 0.01-0.5%
         The ingredients are combined and heated to 75° C. with stirring until the sodium stearate has dissolved. The resulting mixture is cooled to 40° C. followed by addition of a composition of this invention.
   ix. Lotion including Body Lotion, Facial Lotion, and Hand Lotion
   x. Body Powder
   xi. Hand Sanitizers
   xii. Toiletries
   xiii. Body Spray
   xiv. Shave Cream
   xv. Bath Soak
   xvi. Exfoliating Scrub
k) Personal Care Devices
   i. Facial Tissues
   ii. Cleansing wipes
l) Hair Care Products
   i. Shampoo (liquid and dry powder)
   ii. Hair Conditioner (Rinse out and leave-in)
   iii. Hair Rinses
   iv. Hair Refreshers
   v. Hair styling products, Hair Fixative and styling aids
   vi. Hair combing creams
   vii. Hair wax
   viii. Hair foam, hair gel, nonaerosol pump spary
   ix. Hair Bleaches, Dyes and Colorants
   x. Perming agents
m) Beauty Care
   i. Fine Fragrance—Alcoholic. Compositions and methods for incorporating fragrance capsules into alcoholic fine fragrances are described in U.S. Pat. No. 4,428,869. Alcoholic fine fragrances may contain the following:
      1. Ethanol (1-99%)
      2. Water (0-99%)
      3. A suspending aide including but not limited to: hydroxypropyl cellulose, ethyl cellulose, silica, microcrystalline cellulose, carrageenan, propylene glycol alginate, methyl cellulose, sodium carboxymethyl cellulose or xanthan gum (0.1-4%)
      4. Optionally an emulsifier or an emollient may be included including but not limited to those listed above
   ii. Solid Perfume
   iii. Lipstick/lip balm
   iv. Make-up cleanser
   v. Skin care cosmetic such as foundation, pack, sunscreen, skin lotion, milky lotion, skin cream, emollients, skin whitening
   vi. Make-up cosmetic including manicure, mascara, eyeliner, eye shadow, liquid foundation, powder foundation, lipstick and cheek rouge
n) Confectionaries confectionery, preferably selected from the group consisting of chocolate, chocolate bar products, other products in bar form, fruit gums, hard and soft caramels and chewing gum
   i. Gum
      1. Gum base (natural latex chicle gum, most current chewing gum bases also presently include elastomers, such as polyvinylacetate (PVA), polyethylene, (low or medium molecular weight) polyisobutene (PIB), polybutadiene, isobutene-isoprene copolymers (butyl rubber), polyvinylethylether (PVE), polyvinylbutyether, copolymers of vinyl esters and vinyl ethers, styrene-butadiene copolymers (styrene-butadiene rubber, SBR), or vinyl elastomers, for example based on vinylacetate/vinyllaurate, vinylacetate/vinylstearate or ethylene/vinylacetate, as well as mixtures of the mentioned elastomers, as described for example in EP 0 242 325, U.S. Pat. No. 4,518,615, U.S. Pat. No. 5,093,136, U.S. Pat. No. 5,266,336, U.S. Pat. No. 5,601,858 or U.S. Pat. No. 6,986,709) 20-25%
      2. Powdered sugar 45-50%
      3. glucose 15-17%
      4. starch syrup 10-13%
      5. plasticizer 0.1%
      6. flavor 0.8-1.2%
         The components described above were kneaded by a kneader according to the foregoing formulation to provide a chewing gum. Encapsulated Flavor or sensate is then added and blended till homogeneous.
   ii. Breath Fresheners
   iii. Orally Dissolvable Strips
   iv. Chewable Candy
   v. Hard Candy
o) Baked products, preferably selected from the group consisting of bread, dry biscuits, cakes and other cookies;
p) snack foods, preferably selected from the group consisting of baked or fried potato chips or potato dough products, bread dough products and corn or peanut-based extrudates;
   i. Potato, tortilla, vegetable or multigrain chips
   ii. Popcorn
   iii. Pretzels
   iv. Extruded stacks
q) Cereal Products preferably selected from the group consisting of breakfast cereals, muesli bars and precooked finished rice products
r) Alcoholic and non-alcoholic beverages, preferably selected from the group consisting of coffee, tea, wine, beverages containing wine, beer, beverages containing beer, liqueurs, schnapps, brandies, sodas containing fruit, isotonic beverages, soft drinks, nectars, fruit and vegetable juices and fruit or vegetable preparations; instant beverages, preferably selected from the group consisting of instant cocoa beverages, instant tea beverages and instant coffee beverages
   i. Ready to drink liquid drinks
   ii. Liquid Drink Concentrates
   iii. Powder Drinks
   iv. Coffee: Instant Cappucino
      1. Sugar 30-40%
      2. Milk Powder 24-35%
      3. Soluble Coffee 20-25%
      4. Lactose 1-15%
      5. Food Grade Emulsifier 1-3%
      6. Encapsulated Volatile Flavor 0.01-0.5%
   v. Tea
   vi. Alcoholic
s) Spice blends and consumer prepared foods
   i. Powder gravy, sauce mixes
   ii. Condiments
   iii. Fermented Products
t) Ready to heat foods: ready meals and soups, preferably selected from the group consisting of powdered soups, instant soups, precooked soups
   i. Soups
   ii. Sauces
   iii. Stews
   iv. Frozen entrees
u) Dairy Products milk products, preferably selected from the group consisting of milk beverages, ice milk, yogurt, kefir, cream cheese, soft cheese, hard cheese, powdered milk, whey, butter, buttermilk and partially or fully hydrolyzed milk protein-containing products Flavored milk beverages
   i. Yoghurt
   ii. Ice cream
   iii. Bean Curd
   iv. Cheese
v) Soya protein or other soybean fractions, preferably selected from the group consisting of soya milk and products produced therefrom, soya lecithin-containing preparations, fermented products such as tofu or tempeh or products produced therefrom and soy sauces;
w) meat products, preferably selected from the group consisting of ham, fresh or raw sausage preparations, and seasoned or marinated fresh or salt meat products
x) Eggs or egg products, preferably selected from the group consisting of dried egg, egg white and egg yolk
y) and oil-based products or emulsions thereof, preferably selected from the group consisting of mayonnaise, remoulade, dressings and seasoning preparations
z) fruit preparations, preferably selected from the group consisting of jams, sorbets, fruit sauces and fruit fillings; vegetable preparations, preferably selected from the group consisting of ketchup, sauces, dried vegetables, deep-frozen vegetables, precooked vegetables, vegetables in vinegar and preserved vegetables.

The above-listed applications are all well known in the art. For example, fabric softener systems are described in U.S. Pat. Nos. 6,335,315, 5,674,832, 5,759,990, 5,877,145, 5,574,179; 5,562,849, 5,545,350, 5,545,340, 5,411,671, 5,403,499, 5,288,417, and 4,767,547, 4,424,134. Liquid laundry detergents include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Liquid dish detergents are described in U.S. Pat. Nos. 6,069,122 and 5,990,065. Shampoo and conditioners that can employ the present invention include those described in U.S. Pat. Nos. 6,162,423, 5,968,286, 5,935,561, 5,932,203, 5,837,661, 5,776,443, 5,756,436, 5,661,118, 5,618,523, 5,275,755, 5,085,857, 4,673,568, 4,387,090 and 4,705,681. Automatic Dish Detergents are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562.

All parts, percentages, proportions, and ratios refer to herein and in the claims are by weight unless otherwise indicated.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%.”

The terms “capsule” and “microcapsule” herein are used interchangeably.

The invention is described in greater detail by the following non-limiting examples.

### Example 1: Polyurea and Polyurethane Capsule System

A capsule delivery system of this invention, i.e., DS-1, was prepared following the procedures below. This system contains both polyurea and polyurethane capsules, each of which encapsulates a different core mixture. More specifically, the delivery system was prepared using the three parts described below.

Part 1. Emulsion I-1.

In a first beaker, a first core mixture was prepared by mixing a J'Adore fragrance oil (192 grams) with a NEOBEE M5 oil (48 grams; Stepan, Chicago, Ill.). To the oil phase was added 19.2 grams of LUPRANATE M20 (a polymeric methylene diphenyl diisocyanate-based resin containing multiple isocyanate groups, BASF corporation, Wyandotte, Mich.) to obtain Oil Phase I-1, which was then doped with 2,5-bis(5′-tert-butyl-2-benzoxazolyl)thiophene (“BBOT”), a blue dye for measuring optical properties of capsules (See Wang et al. (2007) J. Luminescence 122-123:268-271; and Yang et al. (1997) Synth. Metals 91:335-336). In a second beaker, 23.75 grams of FLEXAN II (sodium polystyrene sulfonate; 10% solution; as a dispersant) was combined with 190 grams of 1.0% WALOCEL CRT 50000 PA (sodium carboxymethylcellulose; a co-dispersant) and 0.05 wt % 1,4-diazabicyclo[2.2.2]octane (“DABCO;” prepared as a 10 wt % solution in water; a polyurethane catalyst). To this mixture was added the BBOT-doped Oil Phase I-1 described above under high shearing at 6500 rpm for one minute to obtain a first emulsion, i.e., Emulsion I-1.

Part 2. Slurry II-1.

Like in Part 1, a second core mixture in this part was prepared by mixing an Apple fragrance oil (152 grams, International Flavors and Fragrance, Union Beach, N.J.) with NEOBEE M5 oil (38 grams). To the resultant oil phase was added 15.2 grams of LUPRANATE M20. In a separate beaker, an aqueous surfactant solution was prepared by combining 23.75 grams of FLEXAN II (10% solution) and 190 grams of 1% WALOCEL CRT 50000 PA with water (41.8 grams). The oil phase was then emulsified with the aqueous phase to form a second emulsion under shearing at 9500 rpm for three minutes. The second emulsion was placed in a round bottom vessel at 35° C. and 10.26 grams of 40% hexamethylene diamine (“HMDA;” INVISTA, Wichita, Kans.) was added under constant stirring (350 rpm) with an overhead mixer to form a capsule slurry, i.e., Slurry II-1, which was then cured at 55° C. for two hours. About 90 grams of Slurry II-1 was placed in a beaker and residual HMDA was neutralized with 10 wt % HCl thereby quenching the reaction.

Part 3. Polyurea and Polyurethane Fragrance Capsule Delivery System.

Slurry II-1 was added to Emulsion I-1 prepared in Part 1. The resultant mixture was heated to 35° C. Polypropylene glycol, a crosslinking agent, was added with mixing at 350 rpm. Curing at 55° C. for two hours yielded delivery system DS-1.

Optical Microscopy.

DS-1 was analyzed using an optical microscopy. This analysis indicated that two distinct capsules having chemically different capsule walls were present. In addition, both capsules were encapsulated with a different fragrance and an acceptable overall particle size distribution.

### Example 2: Polyurea and Polyurea Capsule System, Simultaneous Curing

Another capsule delivery system of this invention, i.e., DS-2, was prepared following the procedures below.

Part 1. Emulsion I-2.

Apple fragrance oil (80 grams) was mixed with NEOBEE M5 oil (20 grams). To the oil phase was added LUPRANATE M20 (8 grams, a polyfunctional isocyanate). In a separate beaker, an aqueous surfactant solution was prepared by dissolving 5 grams of MORWET D-425 (a sodium salt of naphthalene sulfonate condensate; 25% aqueous solution) in water (87.8 grams). The oil phase and aqueous surfactant solution were mixed and emulsified at 9500 rpm for two minutes to obtain Emulsion I-2.

Part 2. Emulsion II-2.

J'Adore fragrance oil (80 grams) was mixed with NEOBEE M5 oil (20 grams). To this oil phase was added 8 grams of LUPRANATE M20. In a separate beaker, an aqueous surfactant solution was prepared by dissolving 5 grams of MORWET D-425 (25% aqueous solution) in water (87.8 grams). The oil phase and aqueous surfactant solution were mixed and emulsified at 9500 rpm for two minutes to obtain Emulsion II-2.

Part 3. Delivery System DS-2.

A 1000 mL reaction vessel was charged with 18 grams of 40% HMDA aqueous solution. After the reaction vessel was heated to 35° C., Emulsions I-2 and II-2 were added simultaneously with mixing at 350 rpm. Once the addition was complete, the temperature was maintained at 35° C. for 15 minutes to form capsules. Subsequently, the temperature was increased to 55° C. and the capsules were cured for 2 hours to obtain DS-2.

Analytics.

DS-2 was analyzed via optical microscopy. Microscopic data indicated that each fragrance was separately encapsulated in a discernable capsule structure. In particular, microscopic analysis clearly identified the presence of capsules with the fluorescent dye (i.e., capsules with J'Adore) as bright blue spots, whereas those without dye (i.e., capsules with Apple) appeared as empty spheres upon longer exposure times. In addition, free oil analysis indicated that the two fragrances had high encapsulation efficiency. Namely, DS-2 contained J'Adore free oil only at 0.2% and Apple free oil only at 0.1%.

### Example 3: Polyurea/Polyurea Capsule System, Tandem Curing

Two additional capsule delivery systems, i.e., DS-3 and DS-3′, were prepared following the procedures below.

Part 1. Slurry I-3.

Apple fragrance oil (96 grams) was mixed with NEOBEE M5 oil (24 grams). To the oil phase was added 9.6 grams of LUPRANATE M20. In a separate beaker, an aqueous surfactant solution was prepared by dissolving 6 grams of MORWET D-425 (25% aqueous solution) with water (105.4 grams). The oil phase and aqueous phase were emulsified at 9500 rpm for two minutes. After the resultant emulsion was heated to 35° C., 40% HMDA aqueous solution (10.8 grams) was added to obtain polyurea capsules. Subsequently, the temperature was increased to 55° C. and the capsules were cured for 2 hours to obtain Slurry I-3.

Part 2. Emulsion II-3.

J'Adore fragrance oil (96 grams) was mixed with NEOBEE M5 oil (24 grams) and 0.06 grams BBOT organic dye. To the oil phase was added 9.6 grams of LUPRANATE M20. In a separate beaker, an aqueous surfactant solution was prepared by dissolving 6 grams of MORWET D-425 (25% aqueous solution) in water (105.6 grams). The oil phase and aqueous surfactant solution were blended at 6500 rpm for two minutes to give Emulsion II-3.

Part 3. Delivery System DS-3.

The reaction vessel containing Slurry I-3 was charged with Emulsion II-3 and then heated to 35° C. A 40% HMDA aqueous solution (10.8 grams) was added. Subsequently, the temperature was increased to 55° C. and maintained for 2 hours to obtain DS-3.

Analytics.

DS-3 was analyzed via optical microscopy. This analysis was carried out to determine the effect of curing the Apple capsules first and then emulsifying in the J'Adore capsules for a second curing period. Particle size analysis showed differences in particle size indicative of Apple and J'Adore being independently encapsulated in one system. J'Adore capsules were much larger than the Apple capsules, but a few smaller J'Adore capsules were also observed via fluorescent microscopy. In addition, some J'Adore capsules were stuck together in flocks thereby increasing the particle size.

### Example 4: Polyurea/Polyurethane Capsule System, Modulation of Particle Size

A fourth capsule delivery system, DS-4, was prepared following the procedures below.

Part 1. Slurry I-4.

Apple fragrance oil (96 grams) was mixed with NEOBEE M5 oil (24 grams). To the oil phase was added 9.6 grams of LUPRANATE M20. In a separate beaker, an aqueous surfactant solution was prepared by dissolving 6 grams of MORWET D-425 (25% aqueous solution) in water (151 grams). The oil phase and aqueous surfactant solution were emulsified at 13500 rpm for two minutes. After the emulsion was heated to 35° C., a 40% HMDA aqueous solution (10.8 grams) was added. Subsequently, the temperature was increased to 55° C. and maintained at that temperature for 2 hours to obtain Slurry I-4.

Part 2. Emulsion II-4.

J'Adore fragrance oil (96 grams) was mixed with NEOBEE M5 oil (24 grams) and BBOT organic dye (0.15 grams). To the resultant oil phase was added 9.6 grams of LUPRANATE M20. In a separate beaker, an aqueous surfactant solution was prepared by adding 6 grams of MORWET D-425 (25% aqueous solution) and 3 grams of DABCO (10% aqueous solution) to water (148.1 grams). The oil phase and aqueous surfactant solution were mixed and emulsified at 6500 rpm for two minutes to obtain Emulsion II-4.

Part 3. Delivery System DS-4.

The reaction vessel containing Slurry I-4 prepared in Part 1 was charged at 35° C. with Emulsion II-4 prepared in Part 2. Subsequently, the mixture was charged with 8.74 grams of polypropylene glycol and the temperature was increased to 70° C. and maintained at that temperature for 2 hours to yield DS-4.

Analytics.

DS-4 was analyzed via optical microscopy. Microscopic data and particle size distribution analyses indicated that there were two distinct capsule chemistries present. It was observed that the J'Adore capsules were much larger than the Apple capsules. In addition, the J'Adore capsules were to a various degree with smaller capsules, predominantly with Apple capsules. Further, it was observed that the J'Adore capsules had irregular shapes, including larger structures in the range of 50-150 μm in diameter. Both fragrances were encapsulated with low free oil and an acceptable overall particle size distribution.

### Example 5: Melamine Formaldehyde/Melamine Formaldehyde Capsules System

A fifth delivery system, i.e. DS-5, was prepared following the procedure below.

Part I:

A first wall-forming mixture was prepared by mixing in 263.6 g of water Alcapsol (17.1 g, copolymer of acryl amide and acrylic acid, commercially available from BASF, USA) and Cymel (9.2 g, methylated melamine formaldehyde resin, commercially available from Cytec Industries). The pH was adjusted to 5 using acetic acid. Alcapsol and Cymel were allowed to react until the desired viscosity 70-150 cps was reached. A first core material was prepared by mixing 84 g of fragrance oil Jillz (commercially available from International Flavors and Fragrances), 21 g of NEOBEE M5 oil, and 0.05 g of flurorescence dye BBOT. The first core material was added to the first wall-forming mixture. Subsequent high shearing at 9500 rpm on IKA Turrax T-25 yielded a first emulsion. This emulsion was then heated to about 35-45° C. and held at that temperature for 15 minutes to yield a first capsule slurry.

Part II:

A second wall-forming mixture was prepared by mixing the same amount of Alcapsol and Cymel, and a second fragrance core material (84 g of Apple fragrance oil and 21 g of NEOBEE M5 oil). This second mixture was then added to the first capsule slurry prepared in Part I followed by high shear (9500 rpm) emulsification. The high shear rate could be the same as or different from that use in Part I so that the first capsule prepared in Part I and the second capsule prepared in this part had the same or different particle sizes.

Part III:

The resultant mixture containing two different capsules was cured at 90° C. for 1-3 hours and then cooled to room temperature to obtain DS-5.

Analytics:

Microscopy analyses were carried out to confirm the formation of capsules around the Jillz fragrance droplets, indicated by the fluorescence dye, and around the Apple fragrance droplets, which did not have any dye. As mentioned above, different size capsules were observed when different shear rates were used.

Additional gas chromatography analysis was also carried out and showed a 99% encapsulation efficiency for each fragrance.

### Example 6: Silica/Silica Capsule System

A sixth delivery system, i.e. DS-6, was also prepared. See below.

Part I:

A first core material was prepared by mixing fragrance oil Jillz (93 g), NEOBEE M5 oil (23.2 g), and a fluorescence dye (0.05 g). The core material was emulsified with dispersant cetyl trimethyl ammonium chloride (“Cetac,” 2.2 g, commercially available from Stepan, Northfield, Ill. in water (65.2 g). Tetraethyl orthosilicate (“TEOS,” 20.1 g, commercially available from Invista, USA was then added under a rigorous mixing condition to obtain a first capsule slurry, which was then cured at room temperature for 18-24 hours.

Part II:

To the first capsule slurry were added Cetac (2.25 g) and a second core material (93 g of fragrance oil Apple and 23.25 g of NEOBEE M5 oil). TEOS (20.13 g) was subsequently added under a rigorous mixing condition to obtain DS-6 containing the a second capsule slurry, together with the first capsule slurry.

Part III:

Delivery system DS-6 was cured at room temperature for 24 hours.

Analytics:

Microscopy analyses were carried out to confirm the formation of capsules around the Jillz fragrance indicated by the fluorescence dye and the Apple fragrance, which did not have any dye. As mentioned above, different size capsules were observed when different shear rates were used.

Additional gas chromatography analysis was also carried out and showed an encapsulation efficiency of at least 90% for each fragrance.

### Example 7: Melamine Formaldehyde/Polyurea Capsules System

Part I:

A first wall-forming mixture was prepared by mixing in 263.55 g of water Alcapsol (17.1 g) and Cymel (9.2 g). The pH was adjusted to 5 using acetic acid. Alcapsol and Cymel were allowed to react until the desired viscosity 70-150 cps was reached. A first fragrance core material was prepared by mixing 84 g of Jillz fragrance oil (commercially available from International Flavors and Fragrances), 21 g of NEOBEE M5 oil, 0.05 g of flurorescence dye BBOT). The first fragrance core material was added to the first wall-forming mixture. Subsequent high shearing at 9500 rpm on IKA Turrax T-25 yielded a first emulsion. This emulsion was then heated to 90° C., cured for 1 hour and cooled to yield the first capsule slurry.

Part II:

The second step involves preparing the Polyurea Capsules. Apple fragrance oil (96 grams) was mixed with NEOBEE M5 oil (24 grams). To the oil phase was added 9.6 grams of LUPRANATE M20. In a separate beaker, an aqueous surfactant solution was prepared by dissolving 1.5 grams of MORWET D-425 in water (23.5 grams). The oil phase and aqueous surfactant solution were emulsified at 9500 rpm for two minutes. After the emulsion was heated to 35° C., a 40% HMDA aqueous solution (10.8 grams) was added. Subsequently, the temperature was increased to 55° C., held for 2 hours, and cooled to yield a delivery system of this invention, i.e., DS-7.

Analytics:

Microscopy analyses were carried out to confirm the formation of capsules around the Jillz fragrance indicated by the fluorescence dye and the Apple fragrance, which did not have any dye. As indicated above, different size capsules were observed if the high shear rates used were different for the 2 fragrances. Additional gas chromatography analysis was also carried out that suggested encapsulation efficiency for each fragrance was about 85-95%.

### Two Single Fragrance Polyurea Capsules and a Mixture for Comparison

Two single fragrance polyurea capsules were prepared separately for comparison studies. More specifically, to prepare the first polyurea capsule, i.e., Comp-1, apple fragrance oil (192 grams) was mixed with NEOBEE M5 oil (48 grams). To the oil phase was added 19.2 grams of LUPRANATE M20. In a separate beaker, an aqueous surfactant solution was prepared by combining 12 grams of MORWET D-425 with water (311 grams). The oil phase and aqueous surfactant solution were mixed and emulsified at 9500 rpm for three minutes. The emulsion was heated to 35° C. and a 40% HMDA solution (21.6 grams) was added with mixing (350 rpm). Subsequently, the temperature was increased to 55° C. and maintained at that temperature for 2 hours to obtain Comp-1.

Another polyurea capsule, i.e., Comp-2, were prepared following the same procedure except that the J'Adore fragrance oil (192 grams) was used instead of the apple fragrance oil.

Comp-1 and Comp-2 were mixed at a ratio of 1:1 to obtain a third sample for comparison, i.e., Comp-3.

### Example 8: Capsule System Performance in a Fabric Conditioner Base

The performance of polyurea (PU) capsules prepared in Examples 2 (simultaneously cured) and 3 (cured in tandem) were compared with the single fragrance PU capsules Comp-1 and Comp-2, and neat fragrance in a fabric conditioner base. The formulations are shown in Table 3, each with a neat equivalent of 0.5%.

TABLE 3

| | | Mass of | Mass |
|---|---|---|---|
| | | Slurry or | of Base |
| Sample | Description | Oil (grams) | (grams) |
| 1 | Apple Neat¹ | 0.55 (oil) | 109.45 |
| 2 | J′ Adore Neat² | 0.55 (oil) | 109.45 |
| | | 0.28 Apple | |
| | | (oil) | |
| 3 | 1:1 Apple:J′ Adore Neat Blend³ | 0.28 J′ | 109.45 |
| | | Adore (oil) | |
| Comp-1 | PU Apple (Example 1)¹ | 1.72 (slurry) | 108.28 |
| Comp-2 | PU J′ Adore (Example 1)² | 1.72 (slurry) | 108.28 |
| Comp-3 | 1:1 PU Apple:J′ Adore Post-Blend³ | 1.72 (slurry) | 108.28 |
| DS-2 | Simultaneously cured (Example 3)³ | 1.72 (slurry) | 108.28 |
| DS-3 | Tandemly cured (Example 4)³ | 1.72 (slurry) | 108.28 |

¹32% Apple.
²32% J′ Adore.
³16% Apple/16% J′ Adore.

The benefit of the instant capsules was evaluated by conducting a laundry experiment following standard protocols using a US washing machine. Towels were used for the washing experiments with a dose of 110 grams fabric condition per wash. The towels were evaluated either damp or after drying overnight by panel of 17 judges and fragrance intensity was rated from a Labeled Magnitude Scale (LMS) scale ranging from 0 to 35. A numerical value of 5 would suggest the fabric only produced very weak intensity while a value of 30 indicates a strong smell. The results of the damp and dry analyses are presented in Tables 4 and 5, respectively. This analysis indicated that, unexpectedly, Samples 7-9 had a good perceived intensity.

TABLE 4

| | | Perceived Intensity |
|---|---|---|
| Sample | Description | Damp (LMS ± SE) |
| 1 | Apple Neat | 17.62 ± 1.12^{a} |
| 2 | J′ Adore Neat | 17.12 ± 1.11^{a} |
| 3 | 1:1 Apple:J′ Adore Neat Blend | 14.13 ± 1.13^{b} |
| Comp-1 | PU Apple | 5.95 ± 1.23^{c} |
| Comp-2 | PU J′ Adore | 6.03 ± 1.25^{c} |
| Comp-3 | 1:1 PU Apple:J′ Adore Post-Blend | 5.27 ± 1.24^{c} |
| DS-2 | Simultaneously cured | 7.11 ± 1.23^{c} |
| DS-3 | Tandemly cured | 5.94 ± 1.23^{c} |

^{a, b, and c}Mean scores without the same letters are significantly different at p = 0.05.

TABLE 5

| | | Perceived Intensity | |
|---|---|---|---|
| | | Dry (LMS ± SE) | |
| Sample | Description | Pre-Rub | Post-Rub |
| 1 | Apple Neat | 2.29 ± 1.21^{c} | 3.13 ± 1.25^{b} |
| 2 | J′ Adore Neat | 3.32 ± 1.232^{ab} | 3.75 ± 1.23^{b} |
| 3 | 1:1 Apple:J′ Adore | 2.74 ± 1.24^{bc} | 2.95 ± 1.25^{b} |
| | Neat Blend | | |
| Comp-1 | PU Apple | 3.03 ± 1.28^{ab} | 9.82 ± 1.19^{a} |
| Comp-2 | PU J′ Adore | 3.70 ± 1.27^{a} | 11.10 ± 1.13^{a} |
| Comp-3 | 1:1 PU Apple:J′ Adore | 2.71 ± 1.24^{bc} | 10.23 ± 1.13^{a} |
| | Post-Blend | | |
| DS-2 | Simultaneously cured | 3.07 ± 1.29^{ab} | 9.64 ± 1.19^{a} |
| DS-3 | Tandemly cured | 3.22 ± 1.32^{ab} | 10.56 ± 1.16^{a} |

^{a, b, c, ab, and bc}Mean scores without the same letters are significantly different at p = 0.05.

Capsule delivery systems DS-1, DS-2, DS-3, and DS-4 were individually added to one of the formulations described in Examples 7-13 below. Unexpectedly, each showed a good perceived intensity in each of the formulations. The contents of each formulation are described in the following examples.

### Example 9: Clear Deodorant Stick Formulation

An exemplary clear deodorant stick formulation is provided in Table 6.

TABLE 6

| Ingredient | Percentage |
|---|---|
| Water | to 100 |
| Phosphatidylglycerol/Diphosphatidylglycerol | 55 |
| Sodium Stearate | 6 |
| PEG-4 | 15 |
| Antibacterial Agent | 0.1 |

### Example 10: Antiperspirant Emulsion Spray Formulation

In this example, an antiperspirant emulsion spray formulation was prepared using the ingredients provided in Table 7.

TABLE 7

| Ingredient | Percentage |
|---|---|
| Water | to 100 |
| Dimethicone | 6 |
| Aluminum Chlorohydrate | 5-6 |
| EDTA | 0.15 |
| Lauryl PEG-9 Polydimethylsiloxyethyl | 0.3 |
| Dimethicone | |
| Phenoxyethanol | 0.3 |
| Isobutane | 70 |

### Example 11: Antiperspirant Emulsion Roll-On Formulation

An exemplary antiperspirant emulsion roll-on formulation was prepared using the ingredients provided in Table 8.

TABLE 8

| Ingredient | Percentage |
|---|---|
| Water | to 100 |
| Aluminum Chlorohydrate or Aluminum | 32-36 |
| Zirconium Tetrachlorohydrex Gly | |
| Steareth-2, Steareth-20 | 0.5-4 |
| Silica | 1-5 |
| Glycerin | 3-5 |
| Dimethicone | 0.5 |

### Example 12: Antiperspirant Clear Emulsion Stick Formulation

An exemplary antiperspirant clear emulsion stick formulation was prepared using the ingredients provided in Table 9.

TABLE 9

| Ingredient | Percentage |
|---|---|
| Water | to 100 |
| Aluminum Zirconium Tetrachlorohydrex Gly | 20 |
| Stearyl Alcohol | 30 |
| C12-C15 Alkyl Benzoate | 25 |
| Glycine | 7 |
| Dimethicone | 0.07 |

### Example 13: Antiperspirant Opaque Emulsion Stick Formulation

An exemplary antiperspirant opaque emulsion stick formulation was prepared using the ingredients provided in Table 10.

TABLE 10

| Ingredient | Percentage |
|---|---|
| Water | to 100 |
| Aluminum Chlorohydrate | 40 |
| Isopropyl Palmitate | 9 |
| Dimethicone | 5.8 |
| Synthetic Wax | 9 |
| Beheneth-10 | 2 |
| Polyglyceryl-3 Diisosterate | 0.3 |
| Acrylates Copolymer | 0.3 |
| PEG/PPG-18/18 Dimethicone | 2 |
| Phenoxyethanol | 0.5 |
| Pentylene Glycol | 0.5 |
| Cetyl PEG/PPG-10/1 | 2 |
| Dimethicone | |

### Example 14: Deodorant Spray Formulation

An exemplary deodorant spray formulation was prepared using the ingredients provided in Table 11.

TABLE 11

| Ingredient | Percentage |
|---|---|
| Denatured Alcohol | to 100 |
| Polyaminopropyl biguanide stearate | 0.2-0.5 |
| Butane, Isobutane, Propane, 152A | 55 |

### Example 15: Antiperspirant Clear Gel Formulation

An exemplary antiperspirant clear gel formulation was prepared using the ingredients provided in Table 12.

TABLE 12

| Ingredient | Percentage |
|---|---|
| Water | to 100 |
| Aluminum Zirconium Tetrachlorohydrex Gly | 25 |
| Silicone | 40 |
| Phosphatidylglycerol | 10 |
| Emulsifier | 10 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Indeed, to achieve the purpose of preparing a capsule delivery system containing multiple capsules, one skilled in the art can design and prepare a delivery system by using different benefit agents including fragrances and flavors, varying the concentrations of the wall-forming materials, activation agents, and/or catalysts to achieve desirable organoleptic or release profiles in a consumable product. Further, the ratios among the wall-forming materials, activation agents, benefit agents, catalysts, and scavengers can also be determined by a skilled artisan through assays known in the art to prepare delivery systems with desirable properties.

From the above description, a skilled artisan can easily ascertain the essential characteristics of the present invention, and without departing from the spirit and scope thereof, can make various changes and modifications of the invention to adapt it to various usages and conditions. Thus, other embodiments are also within the claims.

1-36. (canceled)

37. A method of preparing a capsule delivery system having two different capsules, the method comprising:
(a) providing a first emulsion containing a first benefit agent and a first capsule wall-forming material;
(b) providing a second emulsion containing a second benefit agent and a second capsule wall-forming material;
(c) mixing the first emulsion and the second emulsion to obtain an emulsion mixture;
(d) causing the formation of a first capsule and a second capsule to obtain a mixed capsule slurry, the first capsule containing the first benefit agent encapsulated by a first capsule wall formed of the first capsule wall-forming material, the second capsule containing the second benefit agent encapsulated by a second capsule wall formed of the second capsule wall-forming material, and the first capsule being different from the second capsule; and
(e) curing the mixed capsule slurry to obtain the capsule delivery system,
wherein the first or second benefit agent, independently, is a fragrance, flavor, malodor counteracting agent, or combination thereof.

38. The method of claim 37, wherein each of the first and second capsule walls, independently, is formed of a polyacrylate, polyurea, polyurethane, polyacrylamide, poly(acrylate-co-acrylamide), starch, silica, gelatin and gum Arabic, poly(melamine-formaldehyde), poly(urea-formaldehyde), or combination thereof.

39. The method of claim 38, wherein the first or second capsule wall, independently, is formed of a polyurea, polyurethane, or combination thereof.

40. The method of claim 39, wherein the first or second capsule wall, independently, is a reaction product between a polyisocyanate and an amine cross-linker, an alcohol cross-linker, or a hybrid cross-linker, the polyisocyanate is an aromatic or aliphatic isocyanate having two or more isocyanate groups, the amine cross-linker has two or more amine groups, the alcohol cross-linker has two or more hydroxyl groups, and the hybrid cross-linker has one or more amine groups and one or more hydroxyl groups.

41. The method of claim 37, wherein the mixed capsule slurry is cured at 55° C. to 130° C.

42. The method of claim 37, further comprising the step of adding a catalyst to the first emulsion, the second emulsion, or the emulsion mixture.

43. The method of claim 37, further comprising the step of adding a malodor counteracting agent to the capsule delivery system.

44. The method of claim 37, further comprising the step of adding a third, fourth, fifth, or sixth emulsion to the emulsion mixture or mixed capsule slurry, and causing the formation of a third, fourth, fifth, or sixth capsule, in which each of these capsules has a size of 0.01 to 1000 microns and contains a benefit agent encapsulated by a capsule wall.

45. A method of preparing a capsule delivery system containing two different capsules, the method comprising:
(a) providing a first capsule slurry including a first capsule, in which the first capsule contains a first benefit agent encapsulated by a first capsule wall that is formed of a first wall-forming material;
(b) providing a second emulsion including a second benefit agent and a second wall-forming material;
(c) mixing the first capsule slurry and the second emulsion to obtain a capsule mixture;
(d) causing the formation of a second capsule to obtain a mixed capsule slurry, in which the second capsule contains the second benefit agent encapsulated by a second capsule wall, the second capsule wall is formed of the second wall-forming material, and the second capsule is different from the first capsule; and
(e) curing the mixed capsule slurry to obtain the capsule delivery system,
wherein the first or second benefit agent, independently, is a fragrance, flavor, malodor counteracting agent, or combination thereof.

46. The method of claim 45, wherein each of the first and second capsule wall, independently, is formed of a polyacrylate, polyurea, polyurethane, polyacrylamide, poly(acrylate-co-acrylamide), silica, gelatin and gum Arabic, poly(melamine-formaldehyde), poly(urea-formaldehyde), or combination thereof.

47. The method of claim 46, wherein the first or second capsule wall, independently, is formed of a polyurea, polyurethane, or combination thereof, the polyurea is a reaction product between a polyisocyanate and an amine cross-linker, an alcohol cross-linker, or a hybrid cross-linker, the polyisocyanate is an aromatic or aliphatic isocyanate having two or more isocyanate groups, the amine cross-linker has two or more amine groups, the alcohol cross-linker has two or more hydroxyl groups, and the hybrid cross-linker has one or more amine groups and one or more hydroxyl groups.

48. The method of claim 45, further comprising, before step (c), the step of curing the first capsule slurry at 55° C. to 130° C.

49. A method of preparing a capsule delivery system containing two different capsules, the method comprising:
(a) providing a first capsule emulsion including a first water phase, a first wall-forming material, and a first core material, in which the first core material has a first benefit agent and is dispersed in the first capsule emulsion as oil droplets having a size of 0.01 to 1000 microns;
(b) adding a second core material and a second wall-forming material to the first capsule emulsion, in which the second core material has a second benefit agent;
(c) emulsifying the second core material to obtain a second capsule emulsion, in which the second core material is dispersed in the second capsule emulsion as oil droplets having a size of 0.01 to 1000 microns;
(d) causing the formation of a first and second capsules to obtain a mixed capsule slurry, in which the first capsule is different from the second capsule, the first capsule contains the first benefit agent encapsulated by a first capsule wall, the first capsule wall is formed of the first wall-forming material, the second capsule contains the second benefit agent encapsulated by a second capsule wall, the second capsule wall is formed of the second wall-forming material; and
(e) curing the mixed capsule slurry to obtain the capsule delivery system,
wherein the first or second benefit agent is a fragrance, flavor, malodor counteracting agent, or combination thereof.

50. The method of claim 49, wherein each of the first and second capsule walls, independently, is formed of a polyacrylate, polyurea, polyurethane, polyacrylamide, poly(acrylate-co-acrylamide), silica, gelatin and gum Arabic, poly(melamine-formaldehyde), poly(urea-formaldehyde), or combination thereof.

51. The method of claim 49, further comprising, before step (c), the step of (b1) adding a second water phase and a second dispersant to the first capsule emulsion.

52. The method of claim 49, further comprising, before step (b), the step of (a1) causing the formation of a first capsule, in which the first capsule contains the first benefit agent encapsulated by a first capsule wall, and the first capsule wall is formed of the first wall-forming material.

53. The method of claim 52, further comprising, after step (a1) and before step (b), the step of (a2) curing the first capsule.

54. A capsule delivery system prepared by a method of claim 37.

55. A consumer product comprising a capsule delivery system of claim 54.

56. The consumer product of claim 55, wherein the consumer product is a personal care product, beauty care product, fabric care product, home care product, or oral care product.
